(19) United States
(12) Patent Application Publication
(10) Pub. No.: US 2010/0304454 A1
(43) Pub. Date: Dec. 2, 2010

(54) NOVEL ARABINOSE-FERMENTING EUKARYOTIC CELLS

(75) Inventor: Johannes Adrianus Maria De Bont, Wageningen (NL)

Correspondence Address: BROWDY AND NEIMARK, P.L.L.C.;624 NINTH STREET, NW, SUITE 300, WASHINGTON, DC, 20001-5303, US

(73) Assignee: ROYAL NEDALCO B.V., Bergen op Zoom (NL)

(21) Appl. No.: 12/669,688

(22) PCT Filed: Jul. 21, 2008
(86) PCT No.: PCT/NL2008/050500

§ 371 Date: Mar. 19, 2010

Related U.S. Application Data
(60) Provisional application No. 60/929,951, filed on Jul. 19, 2007.

(30) Foreign Application Priority Data
Jul. 19, 2007 (EP) 07112791.4

(51) Int. Cl. C12P 7/06; C12N 1/19; C12P 1/02

(52) U.S. Cl. 435/161; 435/254.21; 435/171

(57) ABSTRACT

The present invention relates to eukaryotic cells which have the ability to convert L-arabinose into D-xylulose 5-phosphate. The cells have acquired this ability by transformation with nucleotide sequences coding for an arabinose isomerase, a ribulokinase, and a ribulose-5-P-4-epimerase from a bacterium that belongs to a Clavibacter, Arthrobacter or Gramella genus. The cell preferably is a yeast or a filamentous fungus, more preferably a yeast is capable of anaerobic alcoholic fermentation. The may further comprise one or more genetic modifications that increase the flux of the pentose phosphate pathway, reduce unspecific aldose reductase activity, confer to the cell the ability to directly isomerise xylose into xylulose, increase the specific xylulose kinase activity, increase transport of at least one of xylose and arabinose into the host cell, decrease sensitivity to catabolite repression, increase tolerance to ethanol, osmolarity or organic acids; and/or reduce production of by-products. The cell preferably is a cell that has the ability to produce a fermentation product such as ethanol, lactic acid, 3-hydroxy-propionic acid, acrylic acid, acetic acid, succinic acid, citric acid, amino acids, 1,3-propane-diol, ethylene, glycerol, -lactam antibiotics and cephalosporins. The invention further relates to processes for producing these fermentation products wherein a cell of the invention is used to ferment arabinose into the fermentation products.

### FIELD OF THE INVENTION

The invention relates to the fields of fermentation technology, molecular biology and biofuel production. In particular the invention relates to an eukaryotic cell having the ability to convert L-arabinose into a fermentation product and to a process for producing a fermentation product wherein this cell is used.

### BACKGROUND OF THE INVENTION

Economically viable ethanol production from the hemicellulose fraction of plant biomass requires the simultaneous conversion of both pentoses and hexoses at comparable rates and with high yields. Yeasts, in particular Saccharomyces spp., are the most appropriate candidates for this process since they can grow fast on hexoses, both aerobically and anaerobically. Furthermore they are much more resistant to the toxic environment of lignocellulose hydrolysates than (genetically modified) bacteria. Although wild-type S. cerevisiae strains rapidly ferment hexoses with high efficiency, they cannot grow on nor use pentoses such as D-xylose and L-arabinose. This inspired various studies to expand the substrate range of S. cerevisiae.

EP 1 499 708 discloses the construction of a L-arabinose-fermenting S. cerevisiae strain by overexpression of the bacterial L-arabinose pathway. In the bacterial pathway, the enzymes L-arabinose isomerase (araA), L-ribulokinase (araB), and L-ribulose-5-phosphate 4-epimerase (araD) are involved converting L-arabinose to L-ribulose, L-ribulose-5-P, and D-xylulose-5-P, respectively. Using the Bacillus subtilis araA gene and the Escherichia coli araB, and araD genes, combined with evolutionary engineering, a S. cerevisiae strain capable of aerobic growth on L-arabinose was obtained. The evolved strain was reported to have acquired a mutation in the L-ribulokinase gene (araB), that resulted in a reduced activity of this enzyme. Enhanced transaldolase (TAL1) activity was also reported to be required for L-arabinose fermentation. Moreover, EP 1 499 708 discloses that overexpression of the gene encoding the S. cerevisiae galactose permease (GAL2)—also known to transport arabinose—improved growth on arabinose. However, although the evolved S. cerevisiae strain produced ethanol from arabinose at a low specific production rate of 60-80 mg h⁻¹ (g dry weight)⁻¹ under oxygen-limited conditions, no anaerobic fermentation of arabinose was observed.

Wisselink et al. (2007, AEM Accepts, published online ahead of print on 1 Jun. 2007; Appl. Environ. Microbiol. doi:10.1128/AEM.00177-07) disclose a S. cerevisiae strain obtained by expression of the L-arabinose isomerase (araA), L-ribulokinase (araB), and L-ribulose-5-phosphate 4-epimerase (araD) of the L-arabinose utilization pathway of Lactobacillus plantarum, overexpression of S. cerevisiae genes encoding the enzymes of the non-oxidative pentose-phosphate pathway, and extensive evolutionary engineering. The resulting S. cerevisiae strain exhibits a rate of arabinose consumption of 0.70 g h⁻¹ 14 (g dry weight)⁻¹ and a rate of ethanol production of 0.29 g h⁻¹ (g dry weight)⁻¹ with an ethanol yield of 0.43 g g⁻¹ during anaerobic growth on L-arabinose as sole carbon source.

WO 03/062430 and WO 06/009434 disclose yeast strains able to convert xylose into ethanol. These yeast strains are able to directly isomerise xylose into xylulose. WO 06/096130 discloses yeast strains able to convert xylose and arabinose simultaneously into ethanol.

### DESCRIPTION OF THE INVENTION

### Definitions

### Arabinose Isomerase

The enzyme “arabinose isomerase” (EC 5.3.1.4) is herein defined as an enzyme that catalyses the direct isomerisation of L-arabinose into L-ribulose and vice versa. The enzyme is also known as a L-arabinose ketol-isomerase. Arabinose isomerases of the invention may be further defined by their amino acid sequence as herein described below. Likewise arabinose isomerases may be defined by the nucleotide sequences encoding the enzyme as well as by nucleotide sequences hybridising to a reference (araA) nucleotide sequence encoding a arabinose isomerase as herein described below.

### L-ribulokinase

The enzyme “L-ribulokinase” (EC 2.7.1.16) is herein defined as an enzyme that catalyses the reaction ATP+L-ribulose=ADP+L-ribulose 5-phosphate. A ribulose kinase of the invention may be further defined by its amino acid sequence as herein described below. Likewise a ribulose kinase may be defined by the nucleotide sequences encoding the enzyme as well as by nucleotide sequences hybridising to a reference nucleotide sequence (araB) encoding a xylulose kinase as herein described below.

### L-ribulose-5-phosphate 4-epimerase

The enzyme “L-ribulose-5-phosphate 4-epimerase” (5.1.3.4) is herein defined as an enzyme that catalyses the epimerisation of L-ribulose 5-phosphate into D-xylulose 5-phosphate and vice versa. The enzyme is also known as L-ribulose phosphate 4-epimerase or ribulose phosphate 4-epimerase. A ribulose 5-phosphate 4-epimerase of the invention may be further defined by its amino acid sequence as herein described below. Likewise a ribulose 5-phosphate 4-epimerase may be defined by the nucleotide sequences encoding the enzyme as well as by nucleotide sequences hybridising to a reference nucleotide sequence (araD) encoding a ribulose 5-phosphate 4-epimerase as herein described below.

### D-ribulose 5-phosphate 3-epimerase

The enzyme “D-ribulose 5-phosphate 3-epimerase” (5.1.3.1) is herein defined as an enzyme that catalyses the epimerisation of D-xylulose 5-phosphate into D-ribulose 5-phosphate and vice versa. The enzyme is also known as phosphoribulose epimerase; erythrose-4-phosphate isomerase; phosphoketopentose 3-epimerase; xylulose phosphate 3-epimerase; phosphoketopentose epimerase; ribulose 5-phosphate 3-epimerase; D-ribulose phosphate-3-epimerase; D-ribulose 5-phosphate epimerase; D-ribulose-5-P 3-epimerase; D-xylulose-5-phosphate 3-epimerase; pentose-5-phosphate 3-epimerase; or D-ribulose-5-phosphate 3-epimerase.

### Ribulose 5-phosphate isomerase

The enzyme “ribulose 5-phosphate isomerase” (EC 5.3.1.6) is herein defined as an enzyme that catalyses direct isomerisation of D-ribose 5-phosphate into D-ribulose 5-phosphate and vice versa. The enzyme is also known as phosphopentosisomerase; phosphoriboisomerase; ribose phosphate isomerase; 5-phosphoribose isomerase; D-ribose 5-phosphate isomerase; D-ribose-5-phosphate ketol-isomerase; or D-ribose-5-phosphate aldose-ketose-isomerase.

### Transketolase

The enzyme “transketolase” (EC 2.2.1.1) is herein defined as an enzyme that catalyses the reaction: D-ribose 5-phosphate+D-xylulose 5-phosphate into sedoheptulose 7-phosphate+D-glyceraldehyde 3-phosphate and vice versa. The enzyme is also known as glycolaldehydetransferase or sedoheptulose-7-phosphate:D-glyceraldehyde-3-phosphate glycolaldehydetransferase.

### Transaldolase

The enzyme “transaldolase” (EC 2.2.1.2) is herein defined as an enzyme that catalyses the reaction: sedoheptulose 7-phosphate+D-glyceraldehyde 3-phosphate into D-erythrose 4-phosphate+D-fructose 6-phosphate and vice versa. The enzyme is also known as dihydroxyacetonetransferase; dihydroxyacetone synthase; formaldehyde transketolase; or sedoheptulose-7-phosphate:D-glyceraldehyde-3-phosphate glycerone-transferase. A transaldolase of the invention may be further defined by its amino acid sequence as herein described below.

### Aldose Reductase

The enzyme “aldose reductase” (EC 1.1.1.21) is herein defined as any enzyme that is capable of reducing an aldose to the corresponding alditol and vice versa. In the context of the present invention an aldose reductase may be any unspecific aldose reductase that is native (endogenous) to a host cell of the invention and that is capable of reducing aldopentoses such as arabinose, xylose or xylulose to arabinitol or xylitol, respectively. Unspecific aldose reductases catalyse the reaction: aldose+NAD(P)H+H⁺ [symbol] alditol+NAD(P)⁺. The enzyme has a wide specificity and is also known as aldose reductase; polyol dehydrogenase (NADP⁺); alditol:NADP oxidoreductase; alditol:NADP⁺ 1-oxidoreductase; NADPH-aldopentose reductase; or NADPH-aldose reductase. A particular example of such an unspecific aldose reductase that is endogenous to S. cerevisiae and that is encoded by the GRE3 gene (Träff et al., 2001, Appl. Environ. Microbiol. 67: 5668-74).

### Xylose Isomerase

The enzyme “xylose isomerase” (EC 5.3.1.5) is herein defined as an enzyme that catalyses the direct isomerisation of D-xylose into D-xylulose and vice versa. The enzyme is also known as a D-xylose ketoisomerase. Some xylose isomerases are also capable of catalysing the conversion between D-glucose and D-fructose and are therefore sometimes referred to as glucose isomerase. Xylose isomerases require bivalent cations like magnesium or manganese as cofactor. Xylose isomerases of the invention may be further defined by their amino acid sequence as herein described below. Likewise xylose isomerases may be defined by the nucleotide sequences encoding the enzyme as well as by nucleotide sequences hybridising to a reference nucleotide sequence encoding a xylose isomerase as herein described below. A unit (U) of xylose isomerase activity is herein defined as the amount of enzyme producing 1 nmol of xylulose per minute, under conditions as described by Kuyper et al. (2003, FEMS Yeast Res. 4: 69-78).

### Xylulose Kinase

The enzyme “xylulose kinase” (EC 2.7.1.17) is herein defined as an enzyme that catalyses the reaction ATP+D-xylulose=ADP+D-xylulose 5-phosphate. The enzyme is also known as a phosphorylating xylulokinase, D-xylulokinase or ATP:D-xylulose 5-phosphotransferase.

### Sequence Identity and Similarity

Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The terms “substantially identical”, “substantial identity” or “essentially similar” or “essential similarity” means that two peptide or two nucleotide sequences, when optimally aligned, such as by the programs GAP or BESTFIT using default parameters, share at least a certain percentage of sequence identity as defined elsewhere herein. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). It is clear than when RNA sequences are said to be essentially similar or have a certain degree of sequence identity with DNA sequences, thymine (T) in the DNA sequence is considered equal to uracil (U) in the RNA sequence. Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA or the open-source software Emboss for Windows (current version 2.7.1-07). Alternatively percent similarity or identity may be determined by searching against databases such as FASTA, BLAST, etc.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

### Hybridising Nucleic Acid Sequences

Nucleotide sequences encoding the enzymes of the invention may also be defined by their capability to hybridise with the nucleotide sequences of SEQ ID NO.'s 10-18, respectively, under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

### Operably Linked

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

### Promoter

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most environmental and developmental conditions. An “inducible” promoter is a promoter that is active under environmental or developmental regulation.

### Protein

The terms “protein” or “polypeptide” are used interchangeably and refer to molecules consisting of a chain of amino acids, without reference to a specific mode of action, size, 3-dimensional structure or origin.

### Homologous

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

### Heterologous

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but has been obtained from another cell or synthetically or recombinantly produced. Generally, though not necessarily, such nucleic acids encode proteins that are not normally produced by the cell in which the DNA is transcribed or expressed. Similarly exogenous RNA encodes for proteins not normally expressed in the cell in which the exogenous RNA is present. Heterologous nucleic acids and proteins may also be referred to as foreign nucleic acids or proteins. Any nucleic acid or protein that one of skill in the art would recognize as heterologous or foreign to the cell in which it is expressed is herein encompassed by the term heterologous nucleic acid or protein. The term heterologous also applies to non-natural combinations of nucleic acid or amino acid sequences, i.e. combinations where at least two of the combined sequences are foreign with respect to each other.

### DETAILED DESCRIPTION OF THE INVENTION

In a first aspect the present invention relates to a eukaryotic cell comprising nucleotide sequences as defined in (a), (b) and (c), whereby the expression of the nucleotide sequences confers to the cell the ability to convert L-arabinose into D-xylulose 5-phosphate. Expressly included in the invention are eukaryotic cells that may already have the ability to convert L-arabinose into D-xylulose 5-phosphate (at a low level) and wherein expression of the nucleotide sequences as defined in (a), (b) and (c) increases the cell's ability to convert L-arabinose into D-xylulose 5-phosphate. Preferably, in the cells of the invention, the ability to convert L-arabinose into D-xylulose 5-phosphate is the ability to convert L-arabinose into D-xylulose 5-phosphate through the subsequent reactions of 1) isomerisation of arabinose into ribulose; 2) phosphorylation of ribulose to ribulose 5-phosphate; and, 3) epimerisation of ribulose 5-phosphate into D-xylulose 5-phosphate. Preferably expression of the nucleotide sequences confers to, or increases in the cell the ability to grow on arabinose as sole carbon and/or energy source, more preferably expression of the nucleotide sequences confers to the cell, or increases in the ability to grow on arabinose as sole carbon and/or energy source through conversion of arabinose into D-xylulose 5-phosphate (and further metabolism of D-xylulose 5-phosphate).

The nucleotide sequence (a) preferably is a nucleotide sequence encoding an arabinose isomerase, preferably a L-arabinose isomerase as herein defined above. The nucleotide sequence encoding the arabinose isomerase preferably is selected from the group consisting of:

(i) a nucleotide sequence encoding an arabinose isomerase comprising an amino acid sequence that has at least 60, 70, 80, 90, 95, 98, 99 or 100% sequence identity with at least one of the amino acid sequences of SEQ ID NO's: 1, 2 and 3;
(ii) a nucleotide sequence comprising a nucleotide sequence that has at least 70, 80, 90, 95, 98, 99 or 100% sequence identity with at least one of the nucleotide sequences of SEQ ID NO's: 10, 11 and 12;
(iii) a nucleotide sequence the complementary strand of which hybridises to a nucleotide sequence of (i) or (ii); and,
(iv) a nucleotide sequence the sequences of which differs from the sequence of a nucleotide sequence of (iii) due to the degeneracy of the genetic code.

The nucleotide sequence (b) preferably is a nucleotide sequence encoding a ribulokinase, preferably a L-ribulokinase as herein defined above. The nucleotide sequence encoding the ribulokinase preferably is selected from the group consisting of:

(i) a nucleotide sequence encoding a ribulokinase comprising an amino acid sequence that has at least 55, 60, 70, 80, 90, 95, 98, 99 or 100% sequence identity with at least one of the amino acid sequences of SEQ ID NO's: 4, 5 and 6;
(ii) a nucleotide sequence comprising a nucleotide sequence that has at least 65, 70, 80, 90, 95, 98, 99 or 100% sequence identity with at least one of the nucleotide sequences of SEQ ID NO's: 13, 14 and 15;
(iii) a nucleotide sequence the complementary strand of which hybridises to a nucleotide sequence of (i) or (ii); and,
(iv) a nucleotide sequence the sequences of which differs from the sequence of a nucleotide sequence of (iii) due to the degeneracy of the genetic code.

The nucleotide sequence (c) preferably is a nucleotide sequence encoding a ribulose-5-P-4-epimerase, preferably a L-ribulose-5-P-4-epimerase as herein defined above. The nucleotide sequence encoding the ribulose-5-P-4-epimerase preferably is selected from the group consisting of:

(i) a nucleotide sequence encoding a ribulose-5-P-4-epimerase comprising an amino acid sequence that has at least 55, 60, 70, 80, 90, 95, 98, 99 or 100% sequence identity with at least one of the amino acid sequences of SEQ ID NO's: 7, 8 and 9;
(ii) a nucleotide sequence comprising a nucleotide sequence that has at least 65, 70, 80, 90, 95, 98, 99 or 100% sequence identity with at least one of the nucleotide sequences of SEQ ID NO's: 16, 17 and 18;
(iii) a nucleotide sequence the complementary strand of which hybridises to a nucleotide sequence of (i) or (ii); and,
(iv) a nucleotide sequence the sequences of which differs from the sequence of a nucleotide sequence of (iii) due to the degeneracy of the genetic code.

A nucleotide sequence encoding an arabinose isomerase comprising an amino acid sequence that has at least 60, 70, 80, 90, 95, 98, 99 or 100% sequence identity with at least one of the amino acid sequences of SEQ ID NO's: 1, 2 and 3, preferably encodes an amino acid sequence wherein active site residues, and/or residues involved in metal ion- and/or substrate-binding are conserved. Such residues may be derived by comparison of the amino acid sequences of SEQ ID NO's: 1, 2 and 3 with the crystal structure of the E. coli L-arabinose isomerase (Manjasetty and Chance, 2006, J Mol Biol. 360 (2):297-309). In addition more than 166 amino acid sequences of arabinose isomerases are known in the art. Sequence alignments of SEQ ID NO's: 1, 2 and 3 with these known arabinose isomerase amino acid sequences will indicate conserved regions and amino acid positions, the conservation of which are important for structure and enzymatic activity. These regions and positions will tolerate no or only conservative amino acid substitutions. Amino acid substitutions outside of these regions and positions are unlikely to greatly affect arabinose isomerase activity.

A nucleotide sequence encoding an L-ribulokinase comprising an amino acid sequence that has at least 60, 70, 80, 90, 95, 98, 99 or 100% sequence identity with at least one of the amino acid sequences of SEQ ID NO's: 4, 5 and 6, preferably encodes an amino acid sequence wherein active site residues, and/or residues involved in substrate-binding are conserved. Such residues may be derived by comparison of the amino acid sequences of SEQ ID NO's: 4, 5 and 6 with the crystal structure of the E. coli L-ribulokinase (Lee and Bendet, 1967, Biol Chem. 242 (9):2043-50; Lee et al., 1970, J Biol Chem. 245 (6):1357-61). In addition more than 5000 amino acid sequences of ribulokinases are known in the art. Sequence alignments of SEQ ID NO's: 4, 5 and 6 with these known ribulokinase amino acid sequences will indicate conserved regions and amino acid positions, the conservation of which are important for structure and enzymatic activity. These regions and positions will tolerate no or only conservative amino acid substitutions. Amino acid substitutions outside of these regions and positions are unlikely to greatly affect ribulokinase activity.

A nucleotide sequence encoding a ribulose-5-P-4-epimerase comprising an amino acid sequence that has at least 60, 70, 80, 90, 95, 98, 99 or 100% sequence identity with at least one of the amino acid sequences of SEQ ID NO's: 7, 8 and 9, preferably encodes an amino acid sequence wherein active site residues, residues involved in metal ion- and substrate-binding and/or residues involved in intersubunit interface are conserved. Such residues may be derived by comparison of the amino acid sequences of SEQ ID NO's: 7, 8 and 9 with the crystal structure of the E. coli ribulose-5-P-4-epimerase (Luo et al., 2001, Biochemistry. 40 (49):14763-71) and comparisons with the structurally related aldolases (Kroemer and Schulz, 2002, Acta Crystallogr D Biol Crystallogr. 58 (Pt 5):824-32; Joerger et al., 2000, Biochemistry. 39 (20):6033-41). In addition more than 600 amino acid sequences of ribulose-5-P-4-epimerases and related aldolases are known in the art. Sequence alignments of SEQ ID NO's: 7, 8 and 9 with these known epimerase/aldolase amino acid sequences will indicate conserved regions and amino acid positions, the conservation of which are important for structure and enzymatic activity. These regions and positions will tolerate no or only conservative amino acid substitutions. Amino acid substitutions outside of these regions and positions are unlikely to greatly affect ribulose-5-P-4-epimerase activity.

In accordance with the invention the eukaryotic host cell may comprise any possible combination of at least one nucleotide sequence as defined in (a), at least one nucleotide sequence as defined in (b) and at least one nucleotide sequence as defined in (c). Herein a nucleotide sequence as defined in (a) can be a nucleotide sequence with a percentage of sequence identity as indicated with an amino acid sequences of an arabinose isomerase (araA) of at least one of Clavibacter michiganensis (C), Arthrobacter aurescens (A) and Gramella forsetii (G); a nucleotide sequence as defined in (b) can be a nucleotide sequence with a percentage of sequence identity as indicated with an amino acid sequences of a L-ribulose kinase (araB) of at least one of Clavibacter michiganensis (C), Arthrobacter aurescens (A) and Gramella forsetii (G); and, a nucleotide sequence as defined in (c) can be a nucleotide sequence with a percentage of sequence identity as indicated with an amino acid sequences of an ribulose-5-P-4-epimerase (araD) of at least one of Clavibacter michiganensis (C), Arthrobacter aurescens (A) and Gramella forsetii (G). In particular the following combinations are included in the invention: AAA; AAC; AAG; ACA; ACC; ACG; AGA; AGC; AGG; CAA; CAC; CAG; CCA; CCC; CCG; CGA; CGC; CGG; GAA; GAC; GAG; GCA; GCC; GCG; GGA; GGC; GGG. Herein the first position in each triplet indicates the type of the araA sequence, the second position indicates the type of araB sequence, and the third position indicates the type of araD sequence, whereby the letters “C”, “A” and “G” indicate amino acid sequences with a percentage amino acid identity as indicated to the corresponding enzymes of Clavibacter michiganensis (C), Arthrobacter aurescens (A) and Gramella forsetii (G), respectively.

In a preferred embodiment of the invention, at least one of the nucleotide sequences as defined in (a), (b) and (c) of claim 1 encodes an amino acid sequences that originate from a bacterial genus selected from the group consisting of Clavibacter, Arthrobacter and Gramella, i.e. the amino acid sequence is identical to an amino acid sequence as it naturally occurs in one of these genera. More preferably, at least one of the nucleotide sequences as defined in (a), (b) and (c) of claim 1 encodes an amino acid sequences that originate from a bacterial species selected from the group consisting of Clavibacter michiganensis, Arthrobacter aurescens and Gramella forsetii, i.e. the amino acid sequence is identical to an amino acid sequence as it naturally occurs in one of these species.

To increase the likelihood that the arabinose isomerase, the ribulokinase and the ribulose-5-P-4-epimerase are expressed at sufficient levels and in active form in the cells of the invention, the nucleotide sequence encoding these enzymes, as well as other enzymes of the invention (see below), are preferably adapted to optimise their codon usage to that of the host cell in question. The adaptiveness of a nucleotide sequence encoding an enzyme to the codon usage of a host cell may be expressed as codon adaptation index (CAI). The codon adaptation index is herein defined as a measurement of the relative adaptiveness of the codon usage of a gene towards the codon usage of highly expressed genes in a particular host cell or organism. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Jansen et al., 2003, Nucleic Acids Res. 31 (8):2242-51). An adapted nucleotide sequence preferably has a CAI of at least 0.2, 0.3, 0.4, 0.5, 0.6 or 0.7. Most preferred are the sequences as listed in SEQ ID NO's: 10-18, which have been codon optimised for expression in S. cerevisiae cells.

The cell of the invention, preferably is a cell capable of active or passive pentose (arabinose and xylose) transport into the cell. The cell preferably contains active glycolysis. The cell further preferably contains an endogenous pentose phosphate pathway. The cell further preferably contains enzymes for conversion of arabinose (and xylose), optionally through pyruvate, to a desired fermentation product such as ethanol, lactic acid, 3-hydroxy-propionic acid, acrylic acid, acetic acid, succinic acid, citric acid, amino acids, 1,3-propane-diol, ethylene, glycerol, β-lactam antibiotics and cephalosporins. A particularly preferred cell is a cell that is naturally capable of alcoholic fermentation, preferably, anaerobic alcoholic fermentation. The cell further preferably has a high tolerance to ethanol, a high tolerance to low pH (i.e. capable of growth at a pH lower than 5, 4, or 3) and towards organic acids like lactic acid, acetic acid or formic acid and sugar degradation products such as furfural and hydroxy-methylfurfural, and a high tolerance to elevated temperatures. Any of these characteristics or activities of the cell may be naturally present in the cell or may be introduced or modified by genetic modification, preferably by self cloning or by the methods of the invention described below. A suitable cell is a cultured cell, a cell that may be cultured in fermentation process e.g. in submerged or solid state fermentation. Particularly suitable cells are eukaryotic microorganism like e.g. fungi, however, most suitable for use in the present inventions are yeasts or filamentous fungi.

Yeasts are herein defined as eukaryotic microorganisms and include all species of the subdivision Eumycotina (Alexopoulos, C. J., 1962, In: Introductory Mycology, John Wiley & Sons, Inc., New York) that predominantly grow in unicellular form. Yeasts may either grow by budding of a unicellular thallus or may grow by fission of the organism. Preferred yeasts as host cells belong to the genera Saccharomyces, Kluyveromyces, Candida, Pichia, Schizosaccharomyces, Hansenula, Kloeckera, Schwanniomyces, and Yarrowia. Preferably the yeast is capable of anaerobic fermentation, more preferably anaerobic alcoholic fermentation. Over the years suggestions have been made for the introduction of various organisms for the production of bio-ethanol from crop sugars. In practice, however, all major bio-ethanol production processes have continued to use the yeasts of the genus Saccharomyces as ethanol producer. This is due to the many attractive features of Saccharomyces species for industrial processes, i.e., a high acid-, ethanol- and osmo-tolerance, capability of anaerobic growth, and of course its high alcoholic fermentative capacity. Preferred yeast species as fungal host cells include S. cerevisiae, S. exiguus, S. bayanus, K. lactis, K. marxianus and Schizosaccharomyces pombe.

Filamentous fungi are herein defined as eukaryotic microorganisms that include all filamentous forms of the subdivision Eumycotina. These fungi are characterized by a vegetative mycelium composed of chitin, cellulose, and other complex polysaccharides. The filamentous fungi of the present invention are morphologically, physiologically, and genetically distinct from yeasts. Vegetative growth by filamentous fungi is by hyphal elongation and carbon catabolism of most filamentous fungi is obligately aerobic. Preferred filamentous fungi as host cells belong to the genera Aspergillus, Trichoderma, Humicola, Acremonium, Fusarium, and Penicillium.

In a cell of the invention, the nucleotide sequence as defined in (a), (b) and (c) are preferably operably linked to a promoter that causes sufficient expression of the nucleotide sequences in the cell to confer to the cell the ability to convert L-arabinose into D-xylulose 5-phosphate. Preferably, each of the nucleotide sequence as defined in (a), (b) and (c) is operably linked to a promoter that causes sufficient expression of the nucleotide sequences in the cell to confer to the cell the ability to convert L-arabinose into D-xylulose 5-phosphate. More preferably the promoter(s) cause sufficient expression of the nucleotide sequences confers to the cell the ability to grow on arabinose as sole carbon and/or energy source, most preferably the promoter(s) cause sufficient expression of the nucleotide sequences confers to the cell the ability to grow on arabinose as sole carbon and/or energy source through conversion of arabinose into D-xylulose 5-phosphate (and further metabolism of D-xylulose 5-phosphate). Suitable promoters for expression of the nucleotide sequence as defined in (a), (b) and (c) include promoters that are insensitive to catabolite (glucose) repression and/or that do require xylose for induction. Promoters having these characteristics are widely available and known to the skilled person. Suitable examples of such promoters include e.g. promoters from glycolytic genes such as the phosphofructokinase (PPK), triose phosphate isomerase (TPI), glyceraldehyde-3-phosphate dehydrogenase (GPD, TDH3 or GAPDH), pyruvate kinase (PYK), phosphoglycerate kinase (PGK), glucose-6-phosphate isomerase promoter (PGI1) promoters from yeasts or filamentous fungi; more details about such promoters from yeast may be found in (WO 93/03159). Other useful promoters are ribosomal protein encoding gene promoters, the lactase gene promoter (LAC4), alcohol dehydrogenase promoters (ADH1, ADH4, and the like), the enolase promoter (ENO), the hexose (glucose) transporter promoter (HXT7), and the cytochrome c1 promoter (CYC1). Other promoters, both constitutive and inducible, and enhancers or upstream activating sequences will be known to those of skill in the art. Preferably the promoter that is operably linked to nucleotide sequence as defined in (a), (b) and (c) is homologous to the host cell. It is preferred that for expression of each of the nucleotide sequence as defined in (a), (b) and (c) a different promoter is used. This will improved stability of the expression construct by avoiding homologous recombination between repeated promoter sequences and it avoids competition different copies of the promoter for limiting trans-acting factors.

A cell of the invention further preferably comprises a genetic modification that increases the flux of the pentose phosphate pathway as described in WO 06/009434. In particular, the genetic modification causes an increased flux of the non-oxidative part pentose phosphate pathway. A genetic modification that causes an increased flux of the non-oxidative part of the pentose phosphate pathway is herein understood to mean a modification that increases the flux by at least a factor 1.1, 1.2, 1.5, 2, 5, 10 or 20 as compared to the flux in a strain which is genetically identical except for the genetic modification causing the increased flux. The flux of the non-oxidative part of the pentose phosphate pathway may be measured as described in WO 06/009434.

Genetic modifications that increase the flux of the pentose phosphate pathway may be introduced in the cells of the invention in various ways. These including e.g. achieving higher steady state activity levels of xylulose kinase and/or one or more of the enzymes of the non-oxidative part pentose phosphate pathway and/or a reduced steady state level of unspecific aldose reductase activity. These changes in steady state activity levels may be effected by selection of mutants (spontaneous or induced by chemicals or radiation) and/or by recombinant DNA technology e.g. by overexpression or inactivation, respectively, of genes encoding the enzymes or factors regulating these genes.

In a preferred cell of the invention, the genetic modification comprises overexpression of at least one enzyme of the (non-oxidative part) pentose phosphate pathway. Preferably the enzyme is selected from the group consisting of the enzymes encoding for ribulose-5-phosphate isomerase, ribulose-5-phosphate 3-epimerase, transketolase and transaldolase. Various combinations of enzymes of the (non-oxidative part) pentose phosphate pathway may be overexpressed. E.g. the enzymes that are overexpressed may be at least the enzymes ribulose-5-phosphate isomerase and ribulose-5-phosphate 3-epimerase; or at least the enzymes ribulose-5-phosphate isomerase and transketolase; or at least the enzymes ribulose-5-phosphate isomerase and transaldolase; or at least the enzymes ribulose-5-phosphate 3-epimerase and transketolase; or at least the enzymes ribulose-5-phosphate 3-epimerase and transaldolase; or at least the enzymes transketolase and transaldolase; or at least the enzymes ribulose-5-phosphate 3-epimerase, transketolase and transaldolase; or at least the enzymes ribulose-5-phosphate isomerase, transketolase and transaldolase; or at least the enzymes ribulose-5-phosphate isomerase, ribulose-5-phosphate 3-epimerase, and transaldolase; or at least the enzymes ribulose-5-phosphate isomerase, ribulose-5-phosphate 3-epimerase, and transketolase. In one embodiment of the invention each of the enzymes ribulose-5-phosphate isomerase, ribulose-5-phosphate 3-epimerase, transketolase and transaldolase are overexpressed in the cell of the invention. Preferred is a cell in which the genetic modification comprises at least overexpression of the enzyme transaldolase. More preferred is a cell in which the genetic modification comprises at least overexpression of both the enzymes transketolase and transaldolase as such a host cell is already capable of anaerobic growth on arabinose. In fact, under some conditions we have found that cells overexpressing only the transketolase and the transaldolase already have the same anaerobic growth rate on arabinose as do cells that overexpress all four of the enzymes, i.e. the ribulose-5-phosphate isomerase, ribulose-5-phosphate 3-epimerase, transketolase and transaldolase. Moreover, cells of the invention overexpressing both of the enzymes ribulose-5-phosphate isomerase and ribulose-5-phosphate 3-epimerase are preferred over cells overexpressing only the isomerase or only the 3-epimerase as overexpression of only one of these enzymes may produce metabolic imbalances.

There are various means available in the art for overexpression of enzymes in the cells of the invention. In particular, an enzyme may be overexpressed by increasing the copynumber of the gene coding for the enzyme in the cell, e.g. by integrating additional copies of the gene in the cell's genome, by expressing the gene from an episomal multicopy expression vector or by introducing an episomal expression vector that comprises multiple copies of the gene. The coding sequence used for overexpression of the enzymes preferably is homologous to the host cell of the invention. However, coding sequences that are heterologous to the host cell of the invention may likewise be applied.

Alternatively overexpression of enzymes in the cells of the invention may be achieved by using a promoter that is not native to the sequence coding for the enzyme to be overexpressed, i.e. a promoter that is heterologous to the coding sequence to which it is operably linked. Although the promoter preferably is heterologous to the coding sequence to which it is operably linked, it is also preferred that the promoter is homologous, i.e. endogenous to the cell of the invention. Preferably the heterologous promoter is capable of producing a higher steady state level of the transcript comprising the coding sequence (or is capable of producing more transcript molecules, i.e. mRNA molecules, per unit of time) than is the promoter that is native to the coding sequence, preferably under conditions where arabinose or arabinose and glucose are available as carbon sources, more preferably as major carbon sources (i.e. more than 50% of the available carbon source consists of arabinose or arabinose and glucose), most preferably as sole carbon sources. Suitable promoters in this context include promoters as described above for expression of the nucleotide sequences as defined in (a), (b) and (c).

A further preferred cell of the invention comprises a genetic modification that reduces unspecific aldose reductase activity in the cell. Preferably, unspecific aldose reductase activity is reduced in the host cell by one or more genetic modifications that reduce the expression of or inactivates a gene encoding an unspecific aldose reductase. Preferably, the genetic modifications reduce or inactivate the expression of each endogenous copy of a gene encoding an unspecific aldose reductase that is capable of reducing an aldopentose, including arabinose, xylose and xylulose, in the cell's genome. A given cell may comprise multiple copies of genes encoding unspecific aldose reductases as a result of di-, poly- or aneu-ploidy, and/or a cell may contain several different (iso)enzymes with aldose reductase activity that differ in amino acid sequence and that are each encoded by a different gene. Also in such instances preferably the expression of each gene that encodes an unspecific aldose reductase is reduced or inactivated. Preferably, the gene is inactivated by deletion of at least part of the gene or by disruption of the gene, whereby in this context the term gene also includes any non-coding sequence up- or down-stream of the coding sequence, the (partial) deletion or inactivation of which results in a reduction of expression of unspecific aldose reductase activity in the host cell. A nucleotide sequence encoding an aldose reductase whose activity is to be reduced in the cell of the invention and amino acid sequences of such aldose reductases are described in WO 06/009434 and include e.g. the (unspecific) aldose reductase genes of S. cerevisiae GRE3 gene (Träff et al., 2001, Appl. Environm. Microbiol. 67: 5668-5674) and orthologues thereof in other species.

In a further preferred embodiment, the cell of the invention that has the ability to convert L-arabinose into D-xylulose 5-phosphate expressing in addition has the ability of isomerising xylose to xylulose as e.g. described in WO 03/0624430 and in WO 06/009434. The ability of isomerising xylose to xylulose is preferably conferred to the cell by transformation with a nucleic acid construct comprising a nucleotide sequence encoding a xylose isomerase. Preferably the cell thus acquires the ability to directly isomerise xylose into xylulose. More preferably the cell thus acquires the ability to grow aerobically and/or anaerobically on xylose as sole energy and/or carbon source though direct isomerisation of xylose into xylulose (and further metabolism of xylulose). It is herein understood that the direct isomerisation of xylose into xylulose occurs in a single reaction catalysed by a xylose isomerase, as opposed to the two step conversion of xylose into xylulose via a xylitol intermediate as catalysed by xylose reductase and xylitol dehydrogenase, respectively.

Several xylose isomerases (and their amino acid and coding nucleotide sequences) that may be successfully used to confer to the cell of the invention the ability to directly isomerise xylose into xylulose have been described in the art. These include the xylose isomerase of Piromyces sp. and of other anaerobic fungi that belongs to the families Neocallimastix, Caecomyces, Piromyces, Orpinomyces, or Ruminomyces (WO 03/0624430), the xylose isomerase of the bacterial genus Bacteroides, including e.g. B. thetaiotaomicron (WO 06/009434) and B. fragilis, and the xylose isomerase of the anaerobic fungus Cyllamyces aberensis (US 20060234364). Preferably, a xylose isomerase that may be used to confer to the cell of the invention the ability to directly isomerise xylose into xylulose is a xylose isomerase comprising an amino acid sequence that has at least 70, 75, 80, 83% amino acid identity with the amino acid sequence of SEQ ID NO. 19 or 20.

The cell of the invention that has the ability of isomerising xylose to xylulose further preferably comprises xylulose kinase activity so that xylulose isomerised from xylose may be metabolised to pyruvate. Preferably, the cell contains endogenous xylulose kinase activity. More preferably, a cell of the invention comprises a genetic modification that increases the specific xylulose kinase activity. Preferably the genetic modification causes overexpression of a xylulose kinase, e.g. by overexpression of a nucleotide sequence encoding a xylulose kinase. The gene encoding the xylulose kinase may be endogenous to the cell or may be a xylulose kinase that is heterologous to the cell. A nucleotide sequence that may be used for overexpression of xylulose kinase in the cells of the invention is e.g. the xylulose kinase gene from S. cerevisiae (XKS1) as described by Deng and Ho (1990, Appl. Biochem. Biotechnol. 24-25: 193-199). Another preferred xylulose kinase is a xylose kinase that is related to the xylulose kinase from Piromyces (xylB; see WO 03/0624430). This Piromyces xylulose kinase is actually more related to prokaryotic kinase than to all of the known eukaryotic kinases such as the yeast kinase. The eukaryotic xylulose kinases have been indicated as non-specific sugar kinases, which have a broad substrate range that includes xylulose. In contrast, the prokaryotic xylulose kinases, to which the Piromyces kinase is most closely related, have been indicated to be more specific kinases for xylulose, i.e. having a narrower substrate range. In the cells of the invention, a xylulose kinase to be overexpressed is overexpressed by at least a factor 1.1, 1.2, 1.5, 2, 5, 10 or 20 as compared to a strain which is genetically identical except for the genetic modification causing the overexpression. It is to be understood that these levels of overexpression may apply to the steady state level of the enzyme's activity, the steady state level of the enzyme's protein as well as to the steady state level of the transcript coding for the enzyme.

The cells according to the invention may comprises further genetic modifications that result in one or more of the characteristics selected from the group consisting of (a) increased transport of arabinose and/or xylose into the cell; (b) decreased sensitivity to catabolite repression; (c) increased tolerance to ethanol, osmolarity or organic acids; and, (e) reduced production of by-products. By-products are understood to mean carbon-containing molecules other than the desired fermentation product and include e.g. arabinitol, xylitol, glycerol and/or acetic acid. Any genetic modification described herein may be introduced by classical mutagenesis and screening and/or selection for the desired mutant, or simply by screening and/or selection for the spontaneous mutants with the desired characteristics. Alternatively, the genetic modifications may consist of overexpression of endogenous genes and/or the inactivation of endogenous genes.

Genes the overexpression of which is desired for increased transport of arabinose and/or xylose into the cell are preferably chosen form genes encoding a hexose or pentose transporter. In S. cerevisiae these genes include HXT1, HXT2, HXT4, HXT5, HXT7 and GAL2, of which HXT7, HXT5 and GAL2 are most preferred (see Sedlack and Ho, Yeast 2004; 21: 671-684). Similarly orthologues of these genes in other species may be overexpressed.

Other genes that may be overexpressed in the cells of the invention include genes coding for glycolytic enzymes and/or ethanologenic enzymes such as alcohol dehydrogenases.

Preferred endogenous genes for inactivation include hexose kinase genes e.g. the S. cerevisiae HXK2 gene (see Diderich et al., 2001, Appl. Environ. Microbiol. 67: 1587-1593); the S. cerevisiae MIG1 or MIG2 genes; genes coding for enzymes involved in glycerol metabolism such as the S. cerevisiae glycerol-phosphate dehydrogenase 1 and/or 2 genes; or (hybridising) orthologues of these genes in other species.

Other preferred further modifications of host cells for xylose fermentation are described in van Maris et al. (2006, Antonie van Leeuwenhoek 90:391-418), WO2006/009434, WO2005/023998, WO2005/111214, and WO2005/091733.

Any of the genetic modifications of the cells of the invention as described herein are, in as far as possible, preferably introduced or modified by self cloning genetic modification.

A preferred cell of the invention with one or more of the genetic modifications described above, including modifications obtained by selection of (spontaneous) mutants, has the ability to grow on L-arabinose and optionally xylose as carbon/energy source, preferably as sole carbon source, and preferably under anaerobic conditions. Preferably the cell produces essentially no arabinitol, e.g. the arabinitol produced is below the detection limit or e.g. less than 5, 2, 1, 0.5, or 0.3% of the carbon consumed on a molar basis. Preferably, in case carbon/energy source also includes xylose, the cell produces essentially no xylitol, e.g. the xylitol produced is below the detection limit or e.g. less than 5, 2, 1, 0.5, or 0.3% of the carbon consumed on a molar basis.

A cell of the invention preferably has the ability to grow on L-arabinose as sole carbon/energy source at a rate of at least 0.01, 0.02, 0.05, 0.1, 0.2, 0.25 or 0.3 h⁻¹ under aerobic conditions, or, more preferably, at a rate of at least 0.005, 0.01, 0.02, 0.05, 0.08, 0.1, 0.12, 0.15 or 0.2 h⁻¹ under anaerobic conditions. A cell of the invention preferably has the ability to grow on a mixture of glucose and L-arabinose (in a 1:1 weight ratio) as sole carbon/energy source at a rate of at least 0.01, 0.02, 0.05, 0.1, 0.2, 0.25 or 0.3 h⁻¹ under aerobic conditions, or, more preferably, at a rate of at least 0.005, 0.01, 0.02, 0.05, 0.08, 0.1, 0.12, 0.15 or 0.2 h⁻¹ under anaerobic conditions.

Preferably, a cell of the invention has a specific L-arabinose consumption rate of at least 346, 400, 600, 700, 800, 900 or 1000 mg h⁻¹ (g dry weight)⁻¹. Preferably, a cell of the invention has a yield of fermentation product (such as ethanol) on L-arabinose that is at least 20, 40, 50, 60, 80, 90, 95 or 98% of the cell's yield of fermentation product (such as ethanol) on glucose. More preferably, the modified host cell's yield of fermentation product (such as ethanol) on L-arabinose is equal to the host cell's yield of fermentation product (such as ethanol) on glucose. Likewise, the modified host cell's biomass yield on L-arabinose is preferably at least 55, 60, 70, 80, 85, 90, 95 or 98% of the host cell's biomass yield on glucose. More preferably, the modified host cell's biomass yield on L-arabinose is equal to the host cell's biomass yield on glucose. It is understood that in the comparison of yields on glucose and L-arabinose both yields are compared under aerobic conditions or both under anaerobic conditions.

In another aspect the invention relates to a eukaryotic cell comprising nucleotide sequences as encoding (a′) an arabinose isomerase, (b′) a xylulose kinase, and (c′) a ribulose-5-P-4-epimerase, whereby the expression of the nucleotide sequences confers to the cell the ability to convert L-arabinose into D-xylulose 5-phosphate. In this embodiment the broad substrate specificity of xylulose kinases, in particular eukaryotic xylulose kinases, is exploited to phosphorylate ribulose (and optionally xylulose). Expressly included in also this embodiment of the invention are eukaryotic cells that may already have the ability to convert L-arabinose into D-xylulose 5-phosphate (at a low level) and wherein expression of the nucleotide sequences as defined in (a′), (b′) and (c′) increases the cell's ability to convert L-arabinose into D-xylulose 5-phosphate. Preferably, in the cells of the invention, the ability to convert L-arabinose into D-xylulose 5-phosphate is the ability to convert L-arabinose into D-xylulose 5-phosphate through the subsequent reactions of 1) isomerisation of arabinose into ribulose; 2) phosphorylation of ribulose to ribulose 5-phosphate; and, 3) epimerisation of ribulose 5-phosphate into D-xylulose 5-phosphate. Preferably expression of the nucleotide sequences confers to, or increases in the cell the ability to grow on arabinose as sole carbon and/or energy source, more preferably expression of the nucleotide sequences confers to the cell, or increases in the ability to grow on arabinose as sole carbon and/or energy source through conversion of arabinose into D-xylulose 5-phosphate (and further metabolism of D-xylulose 5-phosphate).

The nucleotide sequence (a′) encoding the arabinose isomerase may be a nucleotide sequence (a) as defined above, however the nucleotide sequence may also encode any other, preferably bacterial, arabinose isomerase, e.g. those from E. coli, Bacillus and Lactobacillus as described in e.g. EP 1499708 and Wisselink et al. (2007, supra). Preferably, the nucleotide sequence encoding the arabinose isomerase comprises an amino acid sequence that has at least 30, 35, 40, 45, or 50% sequence identity with at least one of the amino acid sequences of SEQ ID NO's: 1, 2 and 3.

The nucleotide sequence (b′) encoding a polypeptide with xylulose kinase activity preferably comprises an amino acid sequence having at least 50, 60, 70, 80, 90 or 95% identity with SEQ ID NO. 21.

The nucleotide sequence (c′) encoding the ribulose-5-P-4-epimerase may be a nucleotide sequence (c) as defined above, however the nucleotide sequence may also encode any other, preferably bacterial, ribulose-5-P-4-epimerase, e.g. those from E. coli, Bacillus and Lactobacillus as described in e.g. EP 1499708 and Wisselink et al. (2007, supra). Preferably, the nucleotide sequence encoding the ribulose-5-P-4-epimerase comprises an amino acid sequence that has at least 30, 35, 40, 45, or 50% sequence identity with at least one of the amino acid sequences of SEQ ID NO's: 7, 8 and 9.

The eukaryotic cell comprising the nucleotide sequence encoding an eukaryotic xylulose kinase, in stead of a bacterial ribulose kinase, may the same as the above described cells comprising the nucleotide sequence encoding a bacterial ribulose kinase sequences in all aspects except for the more broadly defined nucleotide sequences (a′) and (c′) and the different nucleotide sequence (b′).

In another aspect the invention relates to a process for producing a fermentation product selected from the group consisting of ethanol, lactic acid, 3-hydroxy-propionic acid, acrylic acid, acetic acid, succinic acid, citric acid, amino acids, 1,3-propane-diol, ethylene, glycerol, β-lactam antibiotics and cephalosporins. The process preferably comprises the steps of: a) fermenting a medium containing a source of arabinose, and optionally xylose, with a cell as defined hereinabove, whereby the cell ferments arabinose, and optionally xylose, to the fermentation product, and optionally, b) recovery of the fermentation product.

In addition to a source of arabinose the carbon source in the fermentation medium may also comprise a source of glucose. The skilled person will further appreciate that the fermentation medium may further also comprise other types of carbohydrates such as e.g. in particular a source of xylose. The sources of arabinose, glucose and xylose may be arabinose, glucose and xylose as such (i.e. as monomeric sugars) or they may be in the form of any carbohydrate oligo- or polymer comprising arabinose, glucose and/or xylose units, such as e.g. lignocellulose, arabinans, xylans, cellulose, starch and the like. For release of arabinose, glucose and/or xylose units from such carbohydrates, appropriate carbohydrases (such as arabinases, xylanases, glucanases, amylases, cellulases, glucanases and the like) may be added to the fermentation medium or may be produced by the modified host cell. In the latter case the modified host cell may be genetically engineered to produce and excrete such carbohydrases. An additional advantage of using oligo- or polymeric sources of glucose is that it enables to maintain a low(er) concentration of free glucose during the fermentation, e.g. by using rate-limiting amounts of the carbohydrases preferably during the fermentation. This, in turn, will prevent repression of systems required for metabolism and transport of non-glucose sugars such as arabinose and xylose. In a preferred process the modified host cell ferments both the arabinose and glucose, and optionally xylose, preferably simultaneously in which case preferably a modified host cell is used which is insensitive to glucose repression to prevent diauxic growth. In addition to a source of arabinose (and glucose) as carbon source, the fermentation medium will further comprise the appropriate ingredient required for growth of the modified host cell. Compositions of fermentation media for growth of eukaryotic microorganisms such as yeasts and filamentous fungi are well known in the art.

The fermentation process may be an aerobic or an anaerobic fermentation process. An anaerobic fermentation process is herein defined as a fermentation process run in the absence of oxygen or in which substantially no oxygen is consumed, preferably less than 5, 2.5 or 1 mmol/L/h, more preferably 0 mmol/L/h is consumed (i.e. oxygen consumption is not detectable), and wherein organic molecules serve as both electron donor and electron acceptors. In the absence of oxygen, NADH produced in glycolysis and biomass formation, cannot be oxidised by oxidative phosphorylation. To solve this problem many microorganisms use pyruvate or one of its derivatives as an electron and hydrogen acceptor thereby regenerating NAD⁺. Thus, in a preferred anaerobic fermentation process pyruvate is used as an electron (and hydrogen acceptor) and is reduced to fermentation products such as ethanol, lactic acid, 3-hydroxy-propionic acid, acrylic acid, acetic acid, succinic acid, citric acid, amino acids, 1,3-propane-diol, ethylene, glycerol, β-lactam antibiotics and cephalosporins. Anaerobic processes of the invention are preferred over aerobic processes because anaerobic processes do not require investments and energy for aeration and in addition, anaerobic processes produce higher product yields than aerobic processes. Alternatively, the fermentation process of the invention may be run under aerobic oxygen-limited conditions. Preferably, in an aerobic process under oxygen-limited conditions, the rate of oxygen consumption is at least 5.5, more preferably at least 6 and even more preferably at least 7 mmol/L/h.

The fermentation process is preferably run at a temperature that is optimal for the modified cells of the invention. Thus, for most yeasts or fungal cells, the fermentation process is performed at a temperature which is less than 42° C., preferably less than 38° C. For yeast or filamentous fungal cells, the fermentation process is preferably performed at a temperature which is lower than 35, 33, 30 or 28° C. and at a temperature which is higher than 20, 22, or 25° C.

Preferably in the fermentation processes of the invention, the cells stably maintain the nucleic acid constructs that confer to the cell the ability of converting arabinose into D-xylulose 5-phosphate, and optionally isomerising xylose to xylulose. Preferably in the process at least 10, 20, 50 or 75% of the cells retain the abilities to convert arabinose into D-xylulose 5-phosphate, and optionally isomerise xylose to xylulose after 50 generations of growth, preferably under industrial fermentation conditions.

A preferred fermentation process according to the invention is a process for the production of ethanol, whereby the process comprises the steps of: a) fermenting a medium containing a source of arabinose, and optionally xylose, with a cell as defined hereinabove, whereby the cell ferments arabinose, and optionally xylose, to ethanol, and optionally, b) recovery of the ethanol. The fermentation medium may further be performed as described above. In the process the volumetric ethanol productivity is preferably at least 0.5, 1.0, 1.5, 2.0, 2.5, 3.0, 5.0 or 10.0 g ethanol per litre per hour. The ethanol yield on arabinose and/or glucose and/or xylose in the process preferably is at least 50, 60, 70, 80, 90, 95 or 98%. The ethanol yield is herein defined as a percentage of the theoretical maximum yield, which, for arabinose, glucose and xylose is 0.51 g. ethanol per g. arabinose, glucose or xylose.

A further preferred fermentation process according to the invention is a process which comprises fermenting a medium containing a source of arabinose and a source of xylose wherein however two separate strains of cells are used, a first strain of cells as defined hereinabove except that cells of the first strain do not have the ability to (directly) isomerise xylose into xylulose, which cells of the first strain ferment arabinose to the fermentation product; and a second strain of cells as defined hereinabove except that cells of the second strain do not have the ability to convert arabinose to xylulose 5-phosphate, which cells of the second strain ferment xylose to the fermentation product. The process optionally comprises the step of recovery of the fermentation product. The cells of the first and second are further as otherwise described hereinabove.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are offered for illustrative purposes only, and are not intended to limit the scope of the present invention in any way.

### DESCRIPTION OF THE FIGURES

FIG. 1

Physical map of plasmid pRS316 GGA showing the three ara genes The most important restriction-enzyme recognition sites used for cloning are indicated.

FIG. 2

Colony PCR on RN1002 and as a negative control on the host strain RN1000. The Fermentas 1 kb ladder is used to control the length of the amplified fragments. On the left side RN1002 and on the right side RN1000 results are shown. All fragment sizes are as expected. Used primers are indicated in Table 1.

### EXAMPLES

### 1. Example 1

### 1.1. Plasmids

1.1.1 araA

For high level of expression of the bacterial araA and araD genes the corresponding expression cassettes are inserted into the 2μ plasmid pAKX002 that already comprises the Piromyces xylA gene linked the S. cerevisiae TPI promoter. The araA expression cassettes is constructed by amplifying the S. cerevisiae TDH3 promoter (P_{TDH3}) with oligo's that allow to link the TDH3 promoter to the 5′ end of the synthetic araA coding sequences of Arthrobacter aurescens (SEQ ID NO. 10), Clavibacter michiganensis (SEQ ID NO. 11) and Gramella forsetii (SEQ ID NO. 12), and amplifying the S. cerevisiae ADH1 terminator with oligo's that allow to link the 3′ end of the synthetic araA coding sequences to the ADH1 terminator (T_{ADH1}). The two fragments are extracted from gel and mixed in roughly equimolar amounts with the fragments of the synthetic araA coding sequences. On this mixture a PCR is performed using the 5′ P_{TDH3} oligo and the 3′ T_{ADH1} oligo. The resulting P_{TDH3}-araA-T_{ADH1} cassette is gel purified, cut at the 5′ and 3′ restriction sites and then ligated into pAKX002, resulting in plasmids pRN-AAaraA, pRN-CMaraA and pRN-GFaraA, respectively.

1.1.2 araD

The three araD constructs are made by first amplifying a truncated version of the S. cerevisiae HXT7 promoter (P_{HXT7}) with oligo's that allow to link the HXT7 promoter to the 5′ end of the synthetic araD coding sequences of Arthrobacter aurescens (SEQ ID NO. 16), Clavibacter michiganensis (SEQ ID NO. 17) and Gramella forsetii (SEQ ID NO. 18), and amplifying the PGI1 terminator with oligo's that allow to link the 3′ end of the synthetic araD coding sequences to the PGI1 terminator region (T_{PGI}). The resulting fragments were extracted from gel and mixed in roughly equimolar amounts with the synthetic araD coding sequences, after which a PCR was performed using the 5′ P_{HXT7} oligo and the 3′ T_{PGI} oligo. The resulting P_{HXT7}-araD-T_{PGI1} cassettes are gel purified, cut at the 5′ and 3′ restriction sites and ligated into pRN-AAaraA, pRN-CMaraA and pRN-GFaraA, respectively, resulting in plasmids pRN-AAaraAD, pRN-CMaraAD and pRN-GFaraAD, respectively.

1.1.3 araB

For the expression of the three bacterial araB genes, the integrational plasmid pRS305 is used (Gietz and Sugino, 1988, Gene 74:527-534). Aside from the bacterial AraB genes, the S. cerevisiae XKS1 gene was also included on this vector. For this, the P_{ADH1}-XKS1-T_{CYC1} containing PvuI fragment from p415ADHXKS was ligated into the PvuI digested vector backbone from the integration plasmid pRS305, resulting in pRN-XKS1. For expression of the bacterial araB genes, three cassettes containing the synthetic araB coding sequences of Arthrobacter aurescens (SEQ ID NO. 13), Clavibacter michiganensis (SEQ ID NO. 14) and Gramella forsetii (SEQ ID NO. 15) genes between the PGI1 promoter (P_{PGI}) and ADH1 terminator (T_{ADH1}) is constructed by PCR amplification. The AraB expression cassettes are made by amplifying the PGI1 promoter with oligonucleotides that allow to link the PGI1 promoter to the 5′ end of the synthetic araB coding sequences, and amplifying the ADH1 terminator with oligo's that allow to link the 3′ end of the synthetic araB coding sequences to the ADH1 terminator (T_{ADH1}). The resulting P_{PGI1}-araB-T_{ADH1} cassettes are gel purified, digested at the 5′ and 3′ restriction sites and are then ligated into pRN-XKS1, to yield plasmids pRN-XKS1-AAaraB, pRN-XKS1-CMaraB and pRN-XKS1-GFaraB, respectively.

### 1.2 Strains

Media for cultivations of Saccharomyces cerevisiae strains, shake flask and fermenter cultivations as well as sequential batch fermentation under aerobic, oxygen-limited and anaerobic conditions were performed as described in Wisselink et al. (2007, AEM Accepts, published online ahead of print on 1 Jun. 2007; Appl. Environ. Microbiol. doi:10.1128/AEM.00177-07).

1.2.1 Derivation of Host Strain RN679 from RWB218

The S. cerevisiae strains in this work are derived from the xylose-fermenting strain RWB217 (Kuyper et al., 2005a, FEMS Yeast Res. 5:399-409): RWB217 has the following genotype: MATA ura3-52 leu2-112 loxP-PTPI::(−266,−1)TAL1 gre3::hphMX pUGPTPI-TKL1 pUGPTPI-RPE1 KanloxP-PTPI::(−?,−1)RKI1 {p415ADHXKS, pAKX002}. Strain RWB218 is obtained by selection of RWB217 for improved growth on D-xylose (Kuyper et al., 2005b, FEMS Yeast Res. 5:925-934) by plating and restreaking on MYD plates. RWB218 is grown non-selectively on YPD in order to facilitate the loss of plasmids pAKX002 and p415ADHXKS1 (Kuyper et al., 2005a, supra), harbouring the URA3 and LEU2 selective markers, respectively. RWB218 is plated on YPD, single colonies are screened for plasmid loss by testing for uracil and leucine auxotrophy. In order to remove a KanMX cassette—still present after integrating the RKI1 overexpression construct (Kuyper et al., 2005a, supra)—a strain from which both plasmids are lost is transformed with pSH47, containing the cre recombinase (Güldener et al., 1996, Nucleic Acids Res., 24:2519-252410). Transformants containing pSH47 are resuspended in YP with 1% D-galactose and incubated for 1 hour at 30° C. Cells are plated on YPD and colonies are screened for loss of the KanMX marker (G418 resistance) and pSH47 (URA3). A strain that has lost both the KanMX marker and the pSH47 plasmid is designated as RN679. The genotype of RN679 is: MATA ura3-52 leu2-112 loxP-PTPI::(−266,−1)TAL1 gre3::hphMX pUGPTPI-TKL1 pUGPTPI-RPE1 KanloxP-PTPI::(−?,−1)RKI1.

### 1.2.2 Transformations of RN679

RN679 is transformed with:

1) pRN-AAaraAD and pRN-XKS1-AAaraB, resulting in strain RN680;
2) pRN-CMaraAD and pRN-XKS1-CMaraB, resulting in strain RN681; and
3) pRN-GFaraAD and pRN-XKS1-GFaraB, resulting in strain RN681.

### 1.2.3 Selection of Strains RN680, RN681 and RN682 for Aerobic Growth on L-Arabinose

Strains RN680, RN681 and RN682 do not grow on solid synthetic medium supplemented with 2% (w/v) L-arabinose (MYA). Therefore, evolutionary engineering is applied for the selection of cells of the strains RN680, RN681 and RN682 with an improved specific growth rate on arabinose. Prior to the selection in synthetic medium supplemented with 2% of arabinose, cells are pre-grown in synthetic medium with galactose, as it is known that galactose-induced S. cerevisiae cells can transport L-arabinose via the galactose permease GAL2p (Kou et al., 1970, J. Bacteriol. 103:671-67817). Galactose-grown cells of strains RN680, RN681, RN682 and control strain RWB218 are transferred to shake flasks containing MY supplemented with 0.1% D-galactose and 2% L-arabinose. After approximately several weeks of cultivation in the single initial shake flask, the cultures of strains RN680, RN681, RN682 IMS0001 show very slow growth after depletion of the galactose, in contrast to the reference strain RWB218 which does not grow after depletion of galactose. Cells of the cultures are next transferred to fresh synthetic medium supplied with 2% of L-arabinose (MYA). After again 1-3 weeks of cultivation in MYA descendants of strains RN680, RN681, RN682 grow with an improved doubling time, whereas strain RWB219 still does not grow. Next cells are sequentially transferred each time an OD660 of 2-3 is reached to fresh MYA with a start OD660 of approximately 0.05 and gradually the specific growth rate of the sequentially transferred cultures increases.

### 1.2.4 Selection of Strains RN680, RN681 and RN682 for Anaerobic Growth on L-Arabinose

To allow for a more gradual transfer to anaerobic conditions, the aerobically evolved strains, as obtained in Example 2.3 above, are first grown under oxygen-limited conditions. As soon as growth is observed under oxygen-limited conditions, the culture is switched to anaerobic conditions in the next batch cycle. Upon arabinose depletion, as indicated by the CO₂ percentage dropping below 0.05% after the CO₂ production peak, a new cycle is initiated by either manual or automated replacement of approximately 90% of the culture with fresh synthetic medium containing 20 g l⁻¹ L-arabinose. In 10-15 cycles, the anaerobic specific growth rate increases as estimated from the CO₂ profile. After 20-25 cycles no significant further increase of the growth rate is noticed. Single colonies are isolated on solid MYA for anaerobically evolved descendants of each of RN680, RN681 and RN682.

### Example 2

### 2.1 Donor Organisms and Genes

As described in Example 1, three donor organisms were selected:
Arthrobacter aurescens (A)
Clavibacter michiganensis (C)
Gramella forsetii (G)

The arabinose genes selected were:
araA: arabinose isomerase EC 3.5.1.4
araB: ribulokinase EC 2.7.1.16
araD: L-ribulose-5-phosphate 4-epimerase EC 5.1.3.4

The 9 genes were synthesized by EXONBIO based on sequences that were optimized for codon usage in yeast by Nextgen Sciences. See sequence listings.

To express the araA gene in Saccharomyces cerevisiae the HXT7 promoter (410 bp) and the PGI1 terminator (329 bp) sequences were used.

To express the araB gene in Saccharomyces cerevisiae the TPI1 promoter (899 bp) and the ADH1 terminator (351 bp) sequences were used.

To express the araD gene in Saccharomyces cerevisiae the TDH3 promoter (686 bp) and the CYC1 terminator (288 bp) sequences were used

The first three nucleotides in front of the ATG were modified into AAA in order to optimize expression.

### 2.2 Host Organism

The yeast host strain was RN1000. This strain is a derivative of strain RWB 218 (Kuyper et al., FEMS Yeast Research 5, 2005, 399-409). The plasmid pAKX002 encoding the Piromyces XylA is lost in RN1000. The genotype of the host strain is: MatA, ura3-52, leu2-112, gre3::hphMX, loxP-Ptpi::TAL1, KanloxP-Ptpi::RKI1, pUGPtpi-TKL1, pUGPtpi-RPE1, {p415 Padh1XKS1Tcyc1-LEU2}

### 2.3 Molecular Techniques Employed in Plasmid Construction

The synthetic genes were amplified using the ‘polymerase chain reaction (PCR)’ technique facilitating cloning. For each reaction two short synthetic oligomers ‘primers’ were used. The one in the ‘forward’ and the other in the ‘reverse’ mode. Constitutive promoter sequences and terminator sequences from Saccharomyces cerevisiae were also amplified using PCR. In Table 1 an overview of all primers used in this study is given. To minimize PCR-induced sequence mistakes, the Finnzymes proofreading enzyme Phusion was used.

The plasmid used to express the ara genes into yeast is pRS316 (Sikorski R. S., Hieter P., “A system of shuttle vectors and yeast host strains designed for efficient manipulation of DNA in Saccharomyces cerevisiae” Genetics 122:19-27 (1989), accession U03442, ATCC77145). This plasmid is a centromeric plasmid (low copynumber in yeast) that has the URA3 gene for selection.

The construction of the pRS316 GGA plasmid is given below. The primers used contained specific restriction-enzyme recognition sites. Construction involved standard molecular biological techniques.

GaraA: promoter cut with NotI and PstI; ORF cut with PstI and XhoI; terminator cut with XhoI and BsiWI.
GaraB: promoter cut with AgeI and XbaI; ORF cut with XbaI and BssHII; terminator cut with BssHII and BsiWI.
AaraD: promoter cut with AgeI and HindIII; ORF cut with HindIII and BamHI; terminator cut with BamHI and XhoI.

TABLE 1
Overview of the primers used in this study.
Explanation code: e.g. DPF = araD promoter Forward,
BTR = araB terminator Reverse and CMDR =
Clavibacter michiganensis araD Reverse.

| | |
|---|---|
| DPF | AAGAGCTCACCGGTTTATCATTATCAATACTGCC |
| DPR | AAGAATTCAAGCTTTATGTGTGTTTATTCGAAACTAAGTTCTTG |
| DTF | AAGAATTCGGATCCCCTTTTCCTTTGTCGA |
| DTR | AACTCGAGCCTAGGAAGCCTTCGAGCGTC |
| AADF | AAAAGCTTAAGAAAATGAGTTCACTTCTGGAGTC |
| AADR | TTGGATCCGACGTCACCTACCGTAAACGTTTTGG |
| CMDF | AAAAGCTTAAGAAAATGTCCACGTATGCCCC |
| CMDR | TTGGATCCGACGTCATTTTAACGCACCTTGCG |
| GFDF | AAAAGCTTAAGAAAATGTCGAGCCAATACAAAGA |
| GFDR | TTGGATCCGACGTCAGTTCTGTCCATAATATGCG |
| BPF | AACCGGTTTCTTCTTCAGATTCCCTC |
| BPR | TTAGATCTCTAGATTTATGTATGTGTTTTTTGTAGT |
| BTF | AAAGATCTGCGCGCGAATTTCTTATGATTTATG |
| BTR | TTAAGCTTCGTACGTGTGGAAGAACGAT |
| AABF | AATCTAGATTAATAAAATGAATACGTCCGAAAACATACCC |
| AABR | TTGCGCGCGACGTCACGCGGACGCCCC |
| CMBF | AATCTAGATTAATAAAATGCCTTCGGCTCCCG |
| CMBR | TTGCGCGCGACGTCAGGCCCTGGCTTCCCTTTTC |
| GFBF | AATCTAGATTAATAAAATGTCGAATTATGTCATCGGG |
| GFBR | TTGCGCGCGACGTCAAACAGCGAATTCGTTC |
| APF | AAGCGGCCGCGGCTACTTCTCGTAGGAAC |
| APR | TTAGATCTGCAGAATTAAAAAAACTTTTTGTTTTTGTG |
| ATF | AAAGATCTCGAGACAAATCGCTCTTAAATATATACC |
| ATR | TTAAGCTTCGTACGTTTTAAACAGTTGATGAGAACC |
| AAAF | AACTGCAGATATCAAAATGCCATCAGCTACCAGC |
| AAAR | TTCTCGAGAGCGCTAAAGACCACCAGCTAGTTTG |
| CMAF | AACTGCAGATATCAAAATGAGCAGAATCACCAC |
| CMAR | TTCTCGAGAGCGTCATAAACCTTGAGCTAACCTATGG |
| GFAF | AACTGCAGATATCAAAATGACAAATTTTGAGAATAAAGAAGTC |
| GFAR | TTCTCGAGAGCGCTACATTCCGTGCTGAAACAAG |

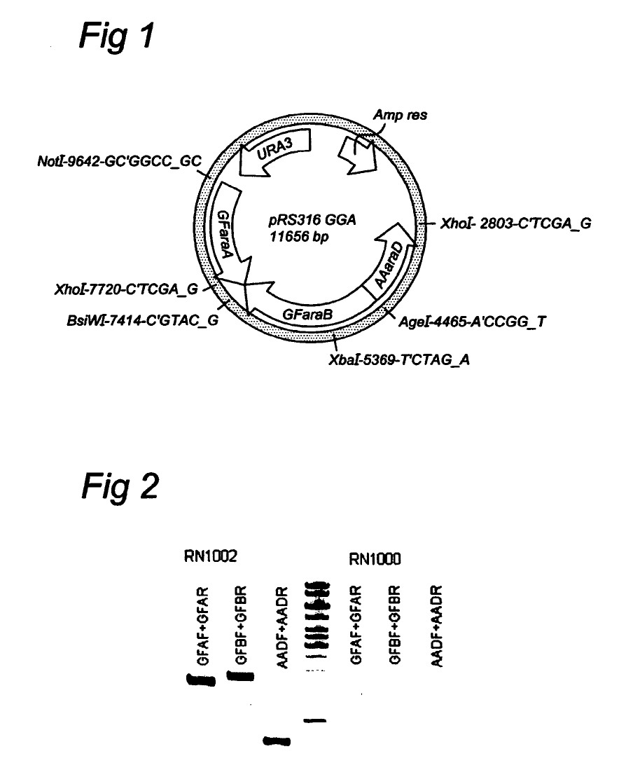

The expression constructs were first assembled per gene and than ligated together into the plasmid pRS316 cut with NotI and XhoI. A and B in opposite direction (adjacent terminator sequences), B and D in opposite direction (adjacent promoter sequences). A physical map of the final plasmid p RS316 GGA is shown in FIG. 1 and its sequence is depicted in SEQ ID NO: 22. Other combinations of AraA, AraB and AraD including the respective promoters were obtained as well and corresponding plasmids were constructed.

### 2.4 Transformation of the Host Organism and Selection of Transformants

RN1000 was transformed with plasmids using the ‘Gietz method’ (Gietz et al., 1992, Nucleic Acids Res. 1992 Mar. 25; 20 (6):1425). Primary selection of transformants was done on mineral medium (YNB+2% glucose) via uracil complementation. Further selection for transformants containing plasmid pRS316 GGA was done on YNB+2% L-arabinose. Colonies emerging on plates of the latter medium grew slowly. However, via Colony PCR it was demonstrated that all three ara genes are present in the transformants (FIG. 2). The yeast transformant thus obtained was designated Royal Nedalco collection number RN1002 and harbours a plasmid with an expression construct for the expression of araA, araB and araD genes.

### 2.5 Oxic Growth of the Engineered Saccharomyces cerevisiae Strain RN1002 at the Expense of L-Arabinose

The purpose of the experiment reported here was to demonstrate that strain RN1002 has the ability to grow at the expense of L-arabinose under oxic (aerobic) conditions.

### 2.5.1 Media

Yeast nitrogen base (YNB, Difco) buffered with 0.17M KH₂PO₄ and 0.72M K₂HPO₄ at pH 5.5 was used for assessing oxic growth at the expense of arabinose. Incubation were performed in the presence of galactose in order to stimulate cell biomass production. After heat sterilization of the medium for 20 min at 120° C., the sugars galactose (0.05%) and/or L-arabinose (1%) were added after filter sterilization.

### 2.5.2 Oxic Cultivation

25 ml YNB with 0.5 g/l galactose with or without 10 g/l L-arabinose was inoculated with material derived from a single colony grown on solid medium (YNB agar with 1% L-arabinose and 0.05% galactose). A culture without any sugar added served as an additional blank. The OD of this culture was below detection level. Cultures where incubated while shaking at 30° C. with oxygen from the air allowed to enter into the liquid medium. The concentrations of L-arabinose and galactose were determined at various times. Cell growth was monitored by measuring the OD.

### 2.5.3 Measurement of the Optical Densities

Optical densities were analyzed by an (Perkin Elmer lambda 2S) spectrophotometer at 700 nm.

### 2.5.4 Determination of Monomeric Sugars

Sugar concentrations in filtered supernatants were determined by high-performance anion-exchange. It was performed on a Dionex system equipped with a CarboPac PA-1 column (4 mm ID×250 mm) in combination with a CarboPac PA guard column (4 mm×50 mm). For the analysis of both L-arabinose and galactose, an isocratic elution (1 ml/min) of 25 minutes was carried out with water. Each elution was followed by a washing and equilibration step. Detection of the compounds was accomplished by the post-column addition of NaOH to the column eluent to raise the pH (>12) before it entered the PAD (Electrochemical detector ED40, Dionex).

### 2.5.5 Results

The results obtained are summarized in Table 2, which demonstrates that strain RN1002 has the ability to metabolize L-arabinose as witnessed by the consumption of L-arabinose and to grow at its expense as demonstrated by the increase in time of OD values of the L-arabinose-containing culture.

### 2.6 Anoxic Production of Ethanol at the Expense of L-Arabinose by the Engineered Saccharomyces cerevisiae Strain RN1002

The purpose of the experiment reported here was to demonstrate that strain RN1002 has the ability to produce ethanol from L-arabinose under anoxic (anaerobic) conditions.

### 2.6.1 Media

For assessing anoxic ethanol production from L-arabinose, a medium containing yeast extract (1% w/w) and peptone (2% w/w) was used. After heat sterilization of the medium for 20 min at 120° C., the sugars galactose (0.5%) and/or arabinose (2%) were added separately after heat sterilization at 110° C.

### 2.6.2 Anoxic Cultivation

To prepare a preculture, strain RN1002 was grown at 32° C. and pH5 in a shake flask culture on 100 ml medium containing yeast extract with peptone and with addition of the sugars galactose (0.5%) and arabinose (2%). After 70 h incubation, this culture was centrifuged twice and cells were resuspended to an OD of 112. This suspension was used to inoculate four anoxic operated stirred fermenters (BAM fermenters purchased from Halotec) with 1 ml each. The subsequent batch fermentations were performed at 32° C. and the working volumes of the four fermentations used in this study were 150 ml each.

### 2.6.3 Gas Analysis

The exhaust gas was cooled by a condenser connected to a cryostat set at 4° C. The exhaust gas flow rate was measured with a Brooks Smart mass flow meter, which is calibrated for CO₂ flow. This mass flow meter was located in a valve box interface (Halotec). The valve box contains all the mechanical parts of the system and its purpose is to control the gas flow of each flask and to house the sensors.

### 2.6.4 Measurement of the Optical Densities

Optical densities were analyzed by an (Perkin Elmer lambda 2S) spectrophotometer at 700 nm.

### 2.6.5 Determination of Ethanol Concentration

Ethanol concentrations in filtered supernatants were determined by HPLC analysis with a Bio-rad Aminex HPX-87H column at 65° C. The column was eluted with 0.25 M sulfuric acid at a flow rate of 0.55 ml min⁻¹.

### 2.6.6 Determination of Monomeric Sugars

Sugar concentrations in filtered supernatants were determined by high-performance anion-exchange. It was performed on a Dionex system equipped with a CarboPac PA-1 column (4 mm ID×250 mm) in combination with a CarboPac PA guard column (4 mm×50 mm). For the analysis of both L-arabinose and galactose, an isocratic elution (1 ml/min) of 25 minutes was carried out with water. Each elution was followed by a washing and equilibration step. Detection of the compounds was accomplished by the post-column addition of NaOH to the column eluent to raise the pH (>12) before it entered the PAD (Electrochemical detector ED40, Dionex).

### 2.6.7 Results

The results obtained are summarized in Table 3 and demonstrate that strain RN1002 has the ability to convert L-arabinose into ethanol.

TABLE 2
Time course of the optical density (A700) and cumulative L-arabinose and
galactose consumption of strain RN1002 during oxic incubations.

| Additions to YNB | Time of | OD | Arabinose | Galactose |
|---|---|---|---|---|
| medium (g/l) | incubation (h) | (A700) | consumed g/l | consumed g/l |
| No addition | 0 | 0.00 | | |
| | 48 | 0.00 | | |
| | 144 | 0.00 | | |
| | 192 | 0.00 | | |
| | 240 | 0.00 | | |
| | 312 | 0.00 | | |
| | 384 | 0.00 | | |
| Galactose (0.5) | 0 | 0.00 | | 0.00 |
| | 48 | 0.98 | | |
| | 144 | 1.24 | | |
| | 192 | 1.02 | | 0.50 |
| Galactose (0.5) + | 0 | 0.01 | 0.00 | 0.00 |
| Arabinose 10) | 48 | 1.42 | | |
| | 144 | 1.51 | | |
| | 192 | 1.44 | 1.14 | 0.50 |
| | 240 | 1.75 | | |
| | 312 | 2.38 | 3.32 | |
| | 384 | 4.08 | 5.26 | |

TABLE 3
Time course of the optical density (A700) and cumulative L-
arabinose and galactose consumption of strain RN1002 during
anoxic incubations as well as the production of ethanol.

| | Time of | | Arabinose | Galactose | Ethanol |
|---|---|---|---|---|---|
| Additions to | incubation | OD | consumed | consumed | produced |
| medium (g/l) | (h) | (A700) | g/l | g/l | (g/l) |
| No addition | 0 | 0.2 | | | 0.00 |
| | 18 | 1.5 | | | 0.00 |
| | 42 | 1.5 | | | 0.00 |
| Arabinose | 0 | 0.2 | 0.00 | | 0.00 |
| (20) | 18 | 2.0 | 0.38 | | 0.25 |
| | 42 | 2.3 | 0.73 | | 0.55 |
| | 66 | 2.3 | 2.20 | | 0.82 |
| Galactose | 0 | 0.2 | | | 0.00 |
| (5) | 18 | 4.2 | | 5.00 | 2.20 |
| | 42 | 4.0 | | | 2.16 |
| Arabinose | 0 | 0.2 | 0.00 | | 0.00 |
| (20) + | 18 | 4.4 | 1.61 | 4.94 | 2.48 |
| Galactose | 42 | 4.4 | 2.59 | 5.01 | 3.01 |
| (5) | 66 | 4.5 | 3.95 | | 3.39 |

1. A eukaryotic cell comprising a first, a second and a third nucleotide sequence the expression of which confers on the cell, or increases in the cell, the ability to convert L-arabinose to D-xylulose 5-phosphate, wherein:
(a) the first nucleotide sequence encodes an arabinose isomerase protein, wherein:
(i) the encoded arabinose isomerase protein comprises an amino acid sequence that is at least 60% identical to at least one of amino acid sequences SEQ ID NO:1, SEQ ID NO:2 and SEQ ID NO:3; or
(ii) the first nucleotide sequence is at least 70% identical to at least one of SEQ ID NO:10, SEQ ID NO:11 and SEQ ID NO:12; or
(iii) the complementary strand of the first nucleotide sequence hybridizes under stringent conditions to the nucleotide sequence of (a)(i) or (a)(ii); or
(iv) the first nucleotide sequence differs from the sequence of (a)(iii) based on degeneracy of the genetic code,
(b) a second nucleotide sequence encoding a ribulokinase protein, wherein:
(i) the encoded ribulokinase protein comprises an amino acid sequence that is at least 55% identical to at least one of amino acid sequences SEQ ID NO:4, SEQ ID NO:5 and SEQ ID NO:6; or
(ii) the second nucleotide sequence is at least 65% identical to at least one of SEQ ID NO:13, SEQ ID NO:14 and SEQ ID NO:15; or
(iii) the complementary strand of the second nucleotide sequence hybridizes under stringent conditions to a nucleotide sequence of (b)(i) or (b)(ii); or
(iv) the second-nucleotide sequence differs from the sequence of b(iii) based on the degeneracy of the genetic code; and
(c) a third nucleotide sequence encoding a ribulose-5-P-4-epimerase protein, wherein:
(i) the third nucleotide sequence encodes a ribulose-5-P-4-epimerase protein comprising an amino acid sequence that is at least 55% identical to at least one of amino acid sequences SEQ ID NO:7, SEQ ID NO:8 and SEQ ID NO:9; or
(ii) the third nucleotide sequence is at least 65% identical to at least one of SEQ ID NO:16, SEQ ID NO:17 and SEQ ID NO:18; or
(iii) complementary strand of the third nucleotide sequence hybridizes under stringent conditions to the nucleotide sequence of (c)(i) or (ii); or
(iv) the third nucleotide sequence differs from the sequence of (c)(iii) based on degeneracy of the genetic code.

2. The cell according to claim 1, wherein at least one of the first, second and third nucleotide sequences encodes an amino acid sequence that originates from a bacterial genus selected from the group consisting of Arthrobacter, Clavibacter, and Gramella.

3. The cell according to claim 1, wherein the first, second and third nucleotide sequence encodes an amino acid sequence that originates from a bacterial species selected from the group consisting of Arthrobacter aurescens, Clavibacter michiganensis, and Gramella forsetii.

4. The cell according to claim 1 which is a yeast or a filamentous fungus of a genus selected from the group consisting of Saccharomyces, Kluyveromyces, Candida, Pichia, Schizosaccharomyces, Hansenula, Kloeckera, Schwanniomyces, Yarrowia, Aspergillus, Trichoderma, Humicola, Acremonium, Fusarium, and Penicillium.

5. The cell according to claim 4, wherein the cell is a yeast cell capable of anaerobic alcoholic fermentation.

6. The cell according to claim 5, wherein the yeast is a member of a species selected from the group consisting of S. cerevisiae, S. exiguus, S. bayanus, K. lactis, K. marxianus and Schizosaccharomyces pombe.

7. The cell according to claim 1, wherein the first, second and third nucleotides sequence are each operably linked to a promoter that causes expression of the nucleotide sequences in the cell at a level that confers upon the cell an ability to convert L-arabinose to D-xylulose 5-phosphate.

8. The cell according to claim 1, that comprises a genetic modification that increases flux of the pentose phosphate pathway.

9. The cell according to claim 8, wherein the genetic modification comprises overexpression of at least one gene of the non-oxidative branch of the pentose phosphate pathway.

10. The cell according to claim 9, wherein the overexpressed gene encodes transaldolase.

11. The cell according to claim 10, wherein the overexpressed genes encode a transketolase and a transaldolase.

12. The cell according to claim 11, wherein the overexpressed genes encode each of a D-ribulose 5-phosphate 3-epimerase, a ribulose 5-phosphate isomerase, a transketolase and a transaldolase.

13. The cell according to claim 1, that comprises a genetic modification that reduces nonspecific aldose reductase activity in the cell.

14. The cell according to claim 13, wherein the genetic modification reduces the expression of, or inactivates, a gene encoding a nonspecific aldose reductase.

15. The cell according to claim 14, whereby the gene is inactivated by at least partial deletion or by disruption of the gene's nucleotide sequence.

16. The cell according to claim 13, wherein expression of each gene that encodes a nonspecific aldose reductase capable of reducing an aldopentose is reduced or said gene is inactivated.

17. The cell according to claim 1, that exhibits an ability to directly isomerize xylose to xylulose.

18. The cell according to claim 17, that further comprises a genetic modification that increases specific xylulose kinase activity.

19. The cell according to claim 18, wherein the genetic modification comprises overexpression of a gene encoding a xylulose kinase.

20. cell according to claim 19, wherein the overexpressed xylulose kinase gene is endogenous to the cell.

21. The cell according to claim 1 that comprises at least one further genetic modification that results in one of the following characteristics:
(a) increased import of xylose or arabinose;
(b) decreased sensitivity to catabolite repression;
(c) increased tolerance to ethanol, osmolarity or organic acids; or
(d) reduced production of by-products.

22. The cell according to claim 1 that expresses one or more enzymes that confer upon the cell the ability to produce at least one fermentation product selected from the group consisting of ethanol, lactic acid, 3-hydroxy-propionic acid, acrylic acid, acetic acid, succinic acid, citric acid, an amino acid, 1,3-propane-diol, ethylene, a glycerol, β-lactam antibiotic and a cephalosporin.

23. A eukaryotic cell comprising a first, second and third nucleotide sequence, the expression of which confers upon the cell an ability, or increases the cell's ability, to convert, L-arabinose to D-xylulose 5-phosphate, wherein the nucleotide sequences are:
(a) the first nucleotide sequence encodes an arabinose isomerase protein;
(b) the second nucleotide sequence encodes a xylulose kinase protein; and,
(c) the third nucleotide sequence encodes a ribulose-5-P-4-epimerase protein.

24. A process for producing a fermentation product, comprising the steps of:
(a) fermenting in a medium containing a source of arabinose the cell according to claim 1, so that the cell ferments arabinose to the fermentation product, and optionally,
(b) recovering the fermentation product,
wherein the fermentation product is ethanol, lactic acid, 3-hydroxy-propionic acid, acrylic acid, acetic acid, succinic acid, citric acid, an amino acid, 1,3-propane-diol, ethylene, glycerol, a β-lactam antibiotic or a cephalosporin.

25. A process for producing a fermentation product, comprising:
(a) fermenting in a medium containing at least one source of xylose and one source of arabinose, the cell according to claim 17, so that the cell ferments at least one of said xylose and arabinose to the fermentation product, and optionally,
(b) recovering the fermentation product,
wherein the fermentation product is ethanol, lactic acid, 3-hydroxy-propionic acid, acrylic acid, acetic acid, succinic acid, citric acid, an amino acid, 1,3-propane-diol, ethylene, glycerol, a β-lactam antibiotic or a cephalosporin.

26. The process according to claim 24, wherein the medium also contains a source of glucose.

27. The process according to claim 24, wherein the fermentation product is ethanol.

28. The process according to claim 27, wherein ethanol productivity is at least 0.5 grams ethanol per liter per hour.

29. The process according to claim 27, wherein ethanol yield is at least 50% of maximal theoretical yield.

30. The process according to claim 24, wherein the process is anaerobic.


SEQUENCE LISTING (52 sequences)

SEQ ID NO: 1 - protein, 511 aa, Arthrobacter aurescens
  misc_feature: araA
MPSATSNPANNTSLEQYEVWFLTGSQHLYGEDVLKQVAAQSQEIANALNANSNVPVKLVWKPVLTDSDAIRRTALEANADDSVIGVTAWMHTFSPAKMWIQGLDALRKPLLHLHTQANRDLPWADIDFDFMNLNQAAHGDREFGYIQSRLGVPRKTVVGHVSNPEVARQVGAWQRASAGWAAVRTLKLTRFGDNMRNVAVTEGDKTEAELRFGVSVNTWSVNELADAVHGAAESDVDSLVAEYERLYEVVPELKKGGARHESLRYSAKIELGLRSFLEANGSAAFTTSFEDLGALRQLPGMAVQRLMADGYGFGAEGDWKTAILVRAAKVMGGDLPGGASLMEDYTYHLEPGSEKILGAHMLEVCPSLTAKKPRVEIHPLGIGGKEDPVRMVFDTDAGPGVVVALSDMRDRFRLVANVVDVVDLDQPLPNLPVARALWEPKPNFATSAAAWLTAGAAHHTVLSTQVGLDVFEDFAEIAKTELLTIDEDTTIKQFKKELNWNAAYYKLAGGL

SEQ ID NO: 2 - protein, 505 aa, Clavibacter michiganensis
  misc_feature: araA
MSRITTSLDHYEVWFLTGSQNLYGEETLQQVAEQSQEIARQLEEASDIPVRVVWKPVLKDSDSIRRMALEANASDGTIGLIAWMHTFSPAKMWIQGLDALQKPFLHLHTQANVALPWSSIDMDFMNLNQAAHGDREFGYIQSRLGVVRKTVVGHVSTESVRASIGTWMRAAAGWAAVHELKVARFGDNMRNVAVTEGDKTEAELKFGVSVNTWGVNDLVARVDAATDAEIDALVDEYETLYDIQPELRRGGERHESLRYGAAIELGLRSFLEEGGFGAFTTSFEDLGGLRQLPGLAVQRLMAEGYGFGAEGDWKTAVLIRAAKVMGSGLPGGASLMEDYTYHLVPGEEKILGAHMLEICPTLTTGRPSLEIHPLGIGGREDPVRLVFDTDPGPAVVVAMSDMRDRFRIVANVVEVVPLDEPLPNLPVARAVWKPAPDLATSAAAWLTAGAAHHTVMSTQVGVEVFEDFAEIARTELLVIDEDTTLKGFTKEVRWNQAYHRLAQGL

SEQ ID NO: 3 - protein, 502 aa, Gramella forsetii
  misc_feature: araA
MTNFENKEVWFITGSQHLYGEETLRQVANNSKEIVEGLNGSDNVPVKLIHQDTVKSSDEITKVMLDANNSSSCIGVILWMHTFSPAKMWIKGLSIIKKPICHFHTQFNAEIPWSKIDMDFMNLNQSAHGDREFGFIMSRMRKKRKVIVGHWKTEVTQKKVGNWQRVALGWDELQHIKVARIGDNMRQVAVTEGDKVAAQIKFGVEVNAYDSSDVTQHIDKVSDDEVNSLLKKYEKDYDLTDALKDGGDQRQSLVDAAKIELGLRAFLEEGGFMAFTDTFENLGALKQLPGLAVQRLMADGYGFGAEGDWKTAALLRAMKVMAQGMEGGTSFMEDYTNHFTEGKDYVLGSHMLEICPSIADSKPTCEVHPLGIGGKEDPVRLVFNSPKGKALNASLVDMGTRFRLIVNEVEAVEPEADLPNLPVARVLWDPKPDMDTAVTAWILAGGAHHTVYTQALSTEFLEDFADIAGIELLVIDDNTSVRQFKDTLNANEAYYHLFQHGM

SEQ ID NO: 4 - protein, 578 aa, Arthrobacter aurescens
  misc_feature: araB
MNTSENIPLDEQFVIGVDYGTLSGRAVVVRVSDGAEIGSGVFEYPHAVVTDNLPGSSQRLPADWALQVPNDYRDVLRNAVPAAVADAGINPENVVGIGTDFTACTMVPTTADGTPLNELERFADRPHAFVKLWRHHAAQPQADRINQLAAERGESWLPRYGGLISSEWEFAKGLQLLEEDPEVYGAMEHWVEAADWIVWQLCGSYVRNACTAGYKGIYQDGKYPSQDFLTALNPDFKDFVSEKLEHTIGRLGDAAGYLTEEAAAWTGLPAGIAVAVGNVDAHVSAPAANAVEPGQLVAIMGTSTCHVMNGDVLREVPGMCGVVDGGIVDGLWGYEAGQSGVGDIFGWFTKNGVPPEYHQAAKDKGLGIHEYLTELAEKQAIGEHGLIALDWHSGNRSVLVDHELSGVVVGQTLATKPEDTYRALLEATAFGTRTIVDAFRDSGVPVKEFIVAGGLLKNKFLMQVYADITGLQLSTIGSEQGPALGSAIHAAVAAGKYKDIREAASSMAAAPGAVYTPIPENVAAYEVLFQEYRTLHDYFGRGTNNVMHRLKAIQRAAIQGSSHNGPAAQASTLEGASA

SEQ ID NO: 5 - protein, 567 aa, Clavibacter michiganensis
  misc_feature: araB
MPSAPVSTATEAQPGADTESYVVGVDYGTLSGRAVVVRVSDGVELGSGVLDYPHAVMDDTLAATGAQLPPEWALQVPSDYVDVLKQAVPAAIREAGIDPARVIGIGTDFTACTMVPTLADGTPLNEVDGYADRPHAYVKLWKHHAAQSHADRINALAEERGEKWLARYGGLISSEWEFAKGLQLLEEDPELYGLMEHWVEAADWIVWQLTGSYVRNACTAGYKGILQDGEYPTAEFLGALNPDFAEFAEEKVAHEIGQLGSAAGTLSAEAAAWTGLPEGIAVAVGNVDAHVTAPVARAVEPGQMVAIMGTSTCHVMNSDVLTEVPGMCGVVDGGIVSGLYGYEAGQSGVGDIFAWYVKNQVPARYAEEAAAAGKSVHQHLTDLAADQPVGGHGLVALDWHSGNRSVLVDHELSGLVIGTTLTTRTEEVYRALLEATAFGTRKIVETFAASGVPVTEFIVAGGLLKNAFLMQAYSDILRLPISVITSEQGPALGSAIHAAVAAGAYPDVRDAGDAMGKVERGKYQPSEERALAYDRLYAEYSTLHDHFGRGANDVMKRLKSLKREARA

SEQ ID NO: 6 - protein, 565 aa, Gramella forsetii
  misc_feature: araB
MSNYVIGLDYGSDSVRAVLVNIDSGKEEASSTHLYKRWKEDKYCEPSINQFRQHPLDHIEGLEKTIKSVLQKTGVEGNSVKAICIDTTGSSPVPVNKDGKALALTEGFEENPNAMMVLWKDHTSINEANEINHLARSWEGEDYTKYEGGIYSSEWFWAKILHIAREDEKVKNAAWSWMEHCDLMTYILIGGSDLESFKRSRCAAGHKAMWHESWGGLPSKDFLSQLDPYLAELKDRLYEKTYTSDEVAGNLSKEWAGKLGLSTECIISVGTFDAHAGAVGAKIDEHSLVRVMGTSTCDIMVARNEEIGKNTVKGICGQVDGSVIPGMIGLEAGQSAFGDVLAWFKDVLSWPLENLVYDSEILAEEQKKKLREEVEDNFIPKLTAQAEKLDLSESMPIALDWVNGRRTPDANQELKSAITNLSLGTKAPHIFNALVNSICFGSKMIVDRFESEGVKINNVIGIGGVARKSAFIMQTLANTLDMPIKVASSDEAPALGAAIYAAVAAGLYPNTIEASKKLGSPFEAEYHPQPEKVKELKKYMAEYRELADFVENKITQKNKQNEFAV

SEQ ID NO: 7 - protein, 235 aa, Arthrobacter aurescens
  misc_feature: araD
MSSLLESIAKVRRDVCDLHAELTRYELVVWTAGNVSGRIPGHDLMVIKPSGVSYDQLTPELMVVTDLYGTPVRGMNTGSAGTVDWGNPELSPSSDTAAHAYVYRHMPEVGGVVHTHSTYATAWAARGEEIPCVLTMMGDEFGGPIPVGPFALIGDDSIGQGIVETLKNSNSPAVLMQNHGPFTIGKSAREAVKAAVMCEEVARTVHISRQLGEPLPIDQAKIESLYKRYQNVYGR

SEQ ID NO: 8 - protein, 236 aa, Clavibacter michiganensis
  misc_feature: araD
MSTYAPEIEVAVARVRSEVSRLHGELVRYGLVVWTGGNVSGRVPGADLFVIKPSGVSYDDLSPENMILCDLDGNVIPDTPGSRNAPSSDTAAHAYVYRNMPEVGGVVHTHSTYAVAWAARREPIPCVITAMADEFGGEIPVGPFAIIGDDSIGRGIVETLTGHRSRAVLMAGHGPFTIGKDAKDAVKAAVMVEDVARTVHISRQLGEPAPLPAEAVDSLFDRYQNVYGQAPQGALK

SEQ ID NO: 9 - protein, 234 aa, Gramella forsetii
  misc_feature: araD
MSSQYKDLKKECYDANMQLNALGLVIYTFGNVSAVDREKEVFAIKPSGVPYKDLKPEDIVILDFDNNVIEGEMRPSSDTKTHAYLYKNWKNIGGIAHTHATYSVAWAQSQKDIPIFGTTHADHLTEDIPCAAPMRDDLIEGNYEHNTGIQILDCFEKKGISYEEVPMVLIGNHGPFTWGKDAAKAVYHSKVLEAVAEMAYLTLQINPEAPRLKDSLIKKHYERKHGKDAYYGQN

SEQ ID NO: 10 - DNA, 1536 bp, Arthrobacter aurescens
  misc_feature: araA
atgccatcagctaccagcaaccctgcaaacaatacatccttggagcagtatgaagtgtggttcttaacgggaagccagcatttatatggggaagacgtattaaagcaagttgctgcccagagtcaagagattgctaacgctttaaatgccaactctaacgttccagttaagttagtctggaagcctgttctgactgatagtgacgccattagaagaactgctctagaagctaatgcggatgattccgttatcggtgtaaccgcatggatgcacacgttctcaccagcaaaaatgtggattcaaggcttggatgctttgaggaagccattgctgcatcttcacactcaggctaatagagatttaccgtgggctgatatagacttcgatttcatgaacctaaaccaggcagcacacggtgatagagaatttggatacattcagtctagattaggagtgcccagaaagaccgtagtcggacacgtgtcaaatccggaagtggctcgtcaagttggggcatggcaaagagccagtgcaggttgggctgctgtgaggacacttaaactgacaagattcggtgataatatgaggaacgtcgctgtcaccgaaggagataaaaccgaggctgaattacgttttggcgtttccgtgaatacttggtccgtcaatgaattggctgatgctgtacatggtgctgctgaatcagatgtagatagcttggtggctgagtacgaaaggttgtatgaagtcgttcctgagctaaagaagggcggtgctcgtcatgagtcgctacgttatagtgctaagatagaactaggcctgagatcgttcctagaagcaaacggctcggcagcttttacaacttcgttcgaagatttaggtgctctaagacaattaccagggatggctgttcaaaggttgatggcggatggatacggttttggtgcagagggtgattggaaaaccgcaattttggttagagcggcgaaggtaatgggtggcgacttgccaggcggtgcatcattgatggaagattacacgtatcacttagagcctggcagtgaaaaaatattaggtgctcacatgctggaggtgtgcccaagcttgaccgctaagaagccaagggttgaaatacaccctcttggtataggaggcaaagaagacccggtgagaatggtgtttgacacagatgcagggcctggagtcgtagttgctttatccgacatgagagacaggtttaggttggtagcaaacgttgtggacgttgtggatttagaccagccattaccaaatctgccagtagctagggccctttgggagccaaagcctaattttgcaacatctgctgctgcatggttaacagcaggtgcagctcatcatactgtactatcaactcaagtcggcttagacgtatttgaggattttgcggaaattgcaaaaaccgaattgcttacgatagatgaggataccacaatcaaacaatttaaaaaggagctaaactggaacgctgcgtactacaaactagctggtggtctttaa

SEQ ID NO: 11 - DNA, 1518 bp, Clavibacter michiganensis
  misc_feature: araA
atgagcagaatcaccacaagcttggatcactacgaagtttggttcttaacaggtagccaaaacctttacggcgaagaaacgctgcaacaagttgctgaacaatcccaagagatcgcgaggcaattagaagaggcatcagacataccggtgagggtagtttggaaacctgtgctaaaagacagcgactcaatcagacgtatggctctagaagcaaacgcatccgatggaacaattgggctgatcgcttggatgcacacattttccccagctaagatgtggatccaaggcttggacgcactacaaaaaccattcttgcatctgcacacacaggcaaacgttgccttgccatggtcttcaatcgacatggattttatgaatttaaatcaagctgcacatggagatagggaattcggatacattcaatccaggttaggtgtggtaagaaagacagtagttggtcacgtttccacggaatcggtccgtgcttcaattggaacatggatgagagcagcagctggttgggccgcggttcatgagttgaaagttgctagatttggcgataacatgagaaatgtcgccgtaaccgaaggggacaaaaccgaagctgaattgaaattcggtgtgtctgtcaacacctggggagtgaatgacttagtggcaagagttgatgctgctacagatgcagagattgatgcattagtcgacgaatatgagaccttgtacgatattcaacccgaactgagaagaggtggagaacgtcatgagtcattaaggtacggagctgctatcgaactaggtctaagatcttttctagaagaaggaggatttggcgcgtttacaacgagttttgaggacctaggtggcttgcgtcaattgccagggttagcggtccagagactaatggctgaaggatacggttttggagctgaaggtgactggaaaactgctgtcttaataagggctgcaaaggtaatgggttcaggtcttcctggcggagcgtccttaatggaagattacacctatcacctggtccctggtgaagagaaaatacttggagcacacatgcttgaaatctgccctactctgacgaccgggagaccatctttagaaattcatcctcttggcataggtggtagagaagaccctgtcagattagttttcgataccgatccaggcccagctgttgttgttgcgatgtcagacatgagggatcgtttccgtatcgtagccaacgttgttgaggtggttccactggacgaacctttgccgaacttacccgttgcgagagccgtctggaagcctgcaccagatttggctacttccgccgctgcctggttgacagcaggtgctgctcatcatacagtcatgagtacccaagtaggagtcgaggtattcgaagatttcgctgagatcgcaaggactgaacttctagtaatcgatgaagatacgacccttaagggatttactaaggaggtgcgttggaatcaggcctaccataggttagctcaaggtttatga

SEQ ID NO: 12 - DNA, 1509 bp, Gramella forsetii
  misc_feature: araA
atgacaaattttgagaataaagaagtctggtttatcaccggatcccagcatctatatggcgaagaaacgttaaggcaagttgctaacaattccaaagaaatagttgaaggtttaaatggctccgataacgtacctgtaaagttaattcaccaagatacggtcaaatcatcggatgagataacaaaagtcatgttagatgcgaacaactcaagttcatgcattggggttattttatggatgcatactttctctccagcaaagatgtggataaaagggttgtctataatcaagaaacctatatgccactttcacacccaatttaatgctgagatcccctggtccaaaattgatatggattttatgaatctgaaccaatcggctcatggcgatagggaatttggattcattatgtcccgtatgaggaagaagaggaaagtaattgtaggccactggaagacagaggttacacaaaagaaagtcggaaattggcaacgtgttgccttgggctgggatgaattgcagcacatcaaggtcgctagaattggggataatatgagacaagtggccgtcaccgaaggagataaagtcgcagcccaaatcaaatttggggtggaagttaatgcttacgactcctctgacgtcacacaacatatcgacaaagtgagcgatgatgaagttaactcactactgaaaaagtatgaaaaagattacgacctgactgacgcactaaaggatggtggcgatcaaagacaaagcttagttgatgctgcgaagattgaattaggactacgtgcgttcttggaagaaggtggtttcatggcattcacagataccttcgaaaatctgggcgcactgaaacaattaccgggtcttgctgtccaacgtttaatggctgatggttatggtttcggagctgaaggtgattggaaaacagcagctctactaagagccatgaaggtcatggcccaaggcatggaaggtgggacatcctttatggaagattacaccaatcattttacggaaggtaaggactatgtgttgggttcacatatgttagaaatatgtcctagtatcgctgacagtaagcctacttgcgaagtccatccgctaggtattggaggcaaagaagatccagtaaggttggtgttcaactcaccgaagggtaaagcactgaatgcatcgcttgttgatatgggaacacgtttcagactaatcgttaacgaagtcgaagccgtggaacctgaagctgatttacctaacttacctgtggcaagggtcttatgggatccaaaaccagacatggatactgctgttaccgcttggatattggcagggggagctcatcatacagtatatactcaagccttatcgactgaatttttggaagattttgccgacatagccggtatagaacttctagtgattgacgacaatacgtcagtaaggcagtttaaggataccttgaatgctaacgaagcatactaccacttgtttcagcacggaatgtag

SEQ ID NO: 13 - DNA, 1737 bp, Arthrobacter aurescens
  misc_feature: araB
atgaatacgtccgaaaacatacccttagacgagcaattcgtaataggggtggactacggaacattatctggccgtgctgtcgttgtcagggtgagtgacggagctgaaatcggatcgggtgtttttgagtacccccatgctgttgtgaccgataacttgccaggttcatctcaaagattgcctgccgattgggccctacaagttccaaacgattaccgtgacgtgttacgtaacgccgttccagctgctgtagctgatgccggtatcaaccccgaaaatgttgttggtattgggaccgactttacagcatgtacgatggtgcccactactgcagatggcacaccgttaaatgagttagagcgttttgccgacagaccccatgctttcgttaaactttggagacatcatgctgctcagcctcaagcagacagaataaaccagttggcagccgaaaggggtgagagttggttaccgcgttatggcggtttaatctcaagtgaatgggagttcgccaaggggctacaactgttggaggaagaccctgaagtttacggcgctatggaacattgggtcgaagcagcagattggatcgtatggcagctttgtggctcatatgtgcgtaatgcttgtacagcaggatacaaggggatttaccaagacggcaaatacccgtcacaggactttctaacagcacttaacccagatttcaaggacttcgtatcggaaaaactggaacataccattggccgtctaggggacgctgctggatacttaaccgaagaagctgctgcttggacgggtctacctgccggtatagcagtggcggttggtaatgttgatgcgcacgtttccgctcctgccgctaacgctgtggaacctggacaacttgtcgcaataatgggtaccagtacgtgtcacgttatgaacggtgacgttttgagggaagttccaggtatgtgtggtgtggttgatggtggcatagttgatggattgtgggggtatgaagctggtcaaagtggtgtcggagatatatttggctggtttactaaaaacggtgttccaccagaatatcatcaagctgccaaggacaaagggttaggtattcacgagtatctgacagaattagccgaaaaacaagcgatcggtgaacacggacttattgctcttgactggcattcaggaaacagatctgtcttggttgatcatgaattatctggggttgtagtcggccagaccctggctactaaacctgaggatacatatagggccttgctggaagcaacagccttcgggaccagaaccattgttgatgcattcagagattcgggagtacctgttaaagaatttatcgtagctggagggctgttaaaaaataaattccttatgcaagtctacgctgacattacagggttacagttatccactattggctctgaacaagggcccgctttaggtagcgcaatccatgctgcagtagctgcagggaagtataaggacattcgtgaagcggctagttccatggctgcggccccaggagctgtatacactccaatcccagaaaacgtcgccgcctacgaagtattattccaagagtacaggacacttcacgattatttcggtagaggcactaataacgtgatgcaccgtttaaaggccattcaaagagcggccattcaaggatccagtcacaatggacccgcagcccaagcaagtaccttggaaggggcgtccgcgtag

SEQ ID NO: 14 - DNA, 1704 bp, Clavibacter michiganensis
  misc_feature: araB
atgccttcggctcccgtgagtacagccacggaagctcaaccgggagctgatacagaatcatacgttgtgggcgtcgattacggcactttgagtggcagagctgttgttgttcgtgtttcggatggtgtcgaattgggttccggtgttcttgactatccacacgctgtgatggatgacacattggccgccacaggtgcgcaattaccaccagaatgggccttgcaagtaccatcagactacgtcgatgttttgaagcaagcagttccagccgcaattagagaggcaggtatagatcccgctagagtcatcggtatcggtactgatttcacagcatgcacgatggtgccaactttggcggatggaactcctttaaacgaagtggatggttacgctgacagaccacacgcatacgtcaaactttggaagcaccacgcagcacagtcacatgcagatagaatcaatgcactagcagaggagaggggagaaaagtggttagcaagatatggcggtctaatatcctcagagtgggagttcgcaaaaggcttgcaactattagaggaagacccagaattatacggcttgatggaacattgggttgaagcagctgactggatcgtttggcaattgacaggttcttatgttagaaacgcctgtacggctggctacaagggtatattacaggatggagagtatcctactgcagagttcttaggcgctcttaatccagacttcgccgaattcgctgaagaaaaagtggcccatgaaattggccaattaggttccgcagcgggtacactaagtgccgaggccgcagcatggacaggtttacctgaaggtatagcagttgcagtgggtaatgttgatgctcacgttactgcgcctgtagcccgtgctgtcgagccaggtcaaatggtagcaatcatgggtacctcgacttgccacgtcatgaactcagatgtcttgaccgaagttccaggtatgtgtggtgtggttgacggtggcattgtttccggcttatatggttatgaggccggtcaatcaggtgtcggtgatatcttcgcatggtatgtaaagaaccaagttccggcacgttacgccgaagaagctgcagcagcaggtaaatctgtgcaccaacacttgacggatttagcagctgaccaaccagtcggtggtcatggattagtcgcattggattggcatagtggcaatagatccgtgttggttgaccatgaattgagcggcctagttataggaacgacattaacaacgcgtactgaggaggtatacagagcattgctggaagcaacagcgtttggcacgcgtaaaatcgtcgaaacattcgccgcgagtggtgtacccgtaaccgaattcattgttgcaggtggtcttctgaagaatgcttttttgatgcaagcttattccgacatcctaagattacccatttcagtaatcacttcggaacaaggccctgctcttggttcggctatccacgcagctgttgctgctggcgcctatcccgacgttagagatgctggtgatgccatgggtaaggtagaaagaggtaaataccaaccttcagaggaaagagctcttgcttacgatagactttatgctgaatatagtacgttgcacgatcatttcggtagaggcgccaatgacgtaatgaagagattgaagtcactgaaaagggaagccagggcctaa

SEQ ID NO: 15 - DNA, 1698 bp, Gramella forsetii
  misc_feature: araB
atgtcgaattatgtcatcgggcttgattacggaagtgactctgttagagcagtgctagttaacattgattccggtaaagaggaagctagttccacccatctatacaagagatggaaggaagacaaatactgtgaaccaagcataaaccagttcagacaacatccgttggatcacatagaagggcttgagaaaactataaaaagtgtgttgcaaaagaccggagttgaaggtaacagtgtgaaagccatatgcatagatactacgggatctagtccagtccctgtcaataaagacggtaaggccctagcactaacagaaggatttgaagaaaatcctaacgcaatgatggtgctgtggaaggatcacacatctatcaacgaggccaatgaaatcaatcaccttgcccgtagttgggaaggtgaagattataccaaatacgaaggaggcatctactcgtcagaatggttttgggccaagattttgcacatcgctcgtgaagatgagaaggtcaagaatgctgcatggtcatggatggaacattgtgacctgatgacatacattttgatcgggggttccgatttagagtcctttaaaaggtccaggtgtgccgcgggacataaggctatgtggcatgagtcttggggaggattacctagcaaagatttcttaagtcaactggatccttacttggccgaattaaaggatagactttatgagaagacatacacgtcagatgaagtagcaggtaatttgagcaaagaatgggctgggaaattagggctttcaactgagtgcatcatctcagttggcacctttgacgcccatgcaggtgcagtaggtgccaaaattgatgaacatagcttagtgcgtgttatgggaacatccacgtgtgacattatggtggcaagaaatgaggagataggtaaaaacacagtcaagggtatctgcggtcaagttgatggttcagtgattcctggtatgatcggactagaagcaggtcaatcagcttttggagacgtgctagcctggttcaaggacgttttgtcctggcctttagagaatctagtttacgattcagaaatactagccgaagagcaaaagaaaaagcttagagaagaagttgaagataatttcattcccaagttaacagcacaagctgagaaattagacttgagtgagtctatgcctattgctcttgattgggtaaatggtcgtcgtacccctgatgccaaccaagaattaaagtctgctattacgaatctatcgttaggtactaaagcaccccatattttcaatgctctagtaaactctatctgtttcggcagtaagatgatagttgataggtttgagtcggaaggcgtcaaaattaacaatgtaataggcataggcggcgtagctaggaagtctgcgtttattatgcagacactagccaacacattagacatgccaatcaaggtcgcaagttccgacgaagcgccagcattgggtgctgctatctacgcagcagtggctgcaggtttgtaccccaatacaatagaagccagtaaaaagttagggtcacctttcgaagctgaataccatccacaacctgagaaagttaaagaacttaagaaatatatggctgaatatagagagttggctgatttcgtggagaacaagataactcagaagaacaagcagaacgaattcgctgtttga

SEQ ID NO: 16 - DNA, 708 bp, Arthrobacter aurescens
  misc_feature: araD
atgagttcacttctggagtctatcgccaaggtcaggagagatgtctgcgacttacacgcagaactgaccagatacgagctggttgtttggactgctggtaatgtatccggtaggattccgggccatgacttaatggtgatcaaacccagtggcgttagctacgatcagttgaccccggaactaatggttgttaccgatctatatgggacgcccgtcagaggtatgaatacgggatcagcaggtacggttgactggggcaatcccgaactaagtcccagttctgacacagctgctcatgcctatgtatatagacatatgcccgaagtgggtggtgtcgtccatacacactctacctatgccacagcatgggctgcaagaggagaagaaattccctgcgttctaactatgatgggagatgagtttgggggtccgattcctgtcggtccttttgcgttaatcggagatgattcaataggccagggaatcgtcgagacactaaagaattcaaactctccggctgtgctaatgcagaaccatgggcccttcactatagggaaaagcgcaagagaggccgtgaaggctgccgttatgtgtgaagaagtggcaaggactgttcacatcagcaggcaattaggagaaccattgcccatcgatcaggctaagattgaatccctgtacaaaaggtaccaaaacgtttacggtaggtag

SEQ ID NO: 17 - DNA, 711 bp, Clavibacter michiganensis
  misc_feature: araD
atgtccacgtatgccccagaaatagaggtcgctgttgctagagtccgttccgaagtaagtaggttacatggtgaactagtcaggtacggactggttgtttggactggtgggaatgtctctggtagagtgcctggcgcagatcttttcgttatcaagccgtccggtgtttcatatgacgacctaagtccggaaaacatgatattgtgcgatctagacgggaacgtaattccagataccccagggtcaagaaacgccccaagtagcgatactgccgcacatgcctatgtttacagaaacatgccggaagtaggcggtgttgtacatacccatagcacatacgctgtagcttgggcagcaaggagagaacctatcccctgcgttattaccgctatggccgatgaattcggtggtgaaattccggtcggtccatttgccataattggcgacgatagtattggtcgtggtatagttgaaaccctgacaggtcacagatcccgtgctgttttaatggcgggtcatggtccattcacaattggtaaagatgccaaggatgcggtgaaggctgcagtaatggtggaggacgtggctagaacggtacacatttcccgtcaattaggagaaccagcacctctaccagctgaagctgttgattccctgttcgatagatatcagaatgtttacggtcaagcaccgcaaggtgcgttaaaatga

SEQ ID NO: 18 - DNA, 705 bp, Gramella forsetii
  misc_feature: araD
atgtcgagccaatacaaagatctgaagaaagaatgctacgatgccaatatgcagttgaacgcgttaggactagtaatatacacttttggcaacgtatctgccgtcgacagagaaaaggaagtattcgcaatcaagccatcaggtgtgccttataaggacttaaagccggaagatatcgtcatcctagatttcgataacaacgtgatcgaaggagaaatgaggccatcatctgatacaaaaacacatgcatacttatacaaaaattggaaaaacatcggaggtattgcccatactcacgcaacctatagtgtcgcatgggctcagtcacagaaggatattccaatattcggtaccacacatgcagatcacttaacagaggacataccatgcgcagctccgatgagagatgatttaatcgaaggaaattacgaacataacacgggcatccagatcctagattgcttcgagaaaaaagggattagctacgaggaagttccgatggtgctaatcggcaatcacggtccgtttacatggggaaaagatgctgcgaaagcagtgtaccactcaaaggttcttgaagctgttgcggaaatggcttatttgaccttgcaaataaatcctgaagcgcccagattgaaagactcactgataaaaaagcactacgagagaaagcatggcaaggacgcatattatggacagaactag

SEQ ID NO: 19 - protein, 437 aa, Piromyces
  misc_feature: xylA
MAKEYFPQIQKIKFEGKDSKNPLAFHYYDAEKEVMGKKMKDWLRFAMAWWHTLCAEGADQFGGGTKSFPWNEGTDAIEIAKQKVDAGFEIMQKLGIPYYCFHDVDLVSEGNSIEEYESNLKAVVAYLKEKQKETGIKLLWSTANVFGHKRYMNGASTNPDFDVVARAIVQIKNAIDAGIELGAENYVFWGGREGYMSLLNTDQKREKEHMATMLTMARDYARSKGFKGTFLIEPKPMEPTKHQYDVDTETAIGFLKAHNLDKDFKVNIEVNHATLAGHTFEHELACAVDAGMLGSIDANRGDYQNGWDTDQFPIDQYELVQAWMEIIRGGGFVTGGTNFDAKTRRNSTDLEDIIIAHVSGMDAMARALENAAKLLQESPYTKMKKERYASFDSGIGKDFEDGKLTLEQVYEYGKKNGEPKQTSGKQELYEAIVAMYQ

SEQ ID NO: 20 - protein, 438 aa, Bacteroides thetaiotaomicron
  misc_feature: XI
MATKEFFPGIEKIKFEGKDSKNPMAFRYYDAEKVINGKKMKDWLRFAMAWWHTLCAEGGDQFGGGTKQFPWNGNADAIQAAKDKMDAGFEFMQKMGIEYYCFHDVDLVSEGASVEEYEANLKEIVAYAKQKQAETGIKLLWGTANVFGHARYMNGAATNPDFDVVARAAVQIKNAIDATIELGGENYVFWGGREGYMSLLNTDQKREKEHLAQMLTIARDYARARGFKGTFLIEPKPMEPTKHQYDVDTETVIGFLKAHGLDKDFKVNIEVNHATLAGHTFEHELAVAVDNGMLGSIDANRGDYQNGWDTDQFPIDNYELTQAMMQIIRNGGLGTGGTNFDAKTRRNSTDLEDIFIAHIAGMDAMARALESAAALLDESPYKKMLADRYASFDGGKGKEFEDGKLTLEDVVAYAKTKGEPKQTSGKQELYEAILNMYC

SEQ ID NO: 21 - protein, 600 aa, Saccharomyces cerevisiae
  misc_feature: XKS1
MLCSVIQRQTREVSNTMSLDSYYLGFDLSTQQLKCLAINQDLKIVHSETVEFEKDLPHYHTKKGVYIHGDTIECPVAMWLEALDLVLSKYREAKFPLNKVMAVSGSCQQHGSVYWSSQAESLLEQLNKKPEKDLLHYVSSVAFARQTAPNWQDHSTAKQCQEFEECIGGPEKMAQLTGSRAHFRFTGPQILKIAQLEPEAYEKTKTISLVSNFLTSILVGHLVELEEADACGMNLYDIRERKFSDELLHLIDSSSKDKTIRQKLMRAPMKNLIAGTICKYFIEKYGFNTNCKVSPMTGDNLATICSLPLRKNDVLVSLGTSTTVLLVTDKYHPSPNYHLFIHPTLPNHYMGMICYCNGSLARERIRDELNKERENNYEKTNDWTLFNQAVLDDSESSENELGVYFPLGEIVPSVKAINKRVIFNPKTGMIEREVAKFKDKRHDAKNIVESQALSCRVRISPLLSDSNASSQQRLNEDTIVKFDYDESPLRDYLNKRPERTFFVGGASKNDAIVKKFAQVIGATKGNFRLETPNSCALGGCYKAMWSLLYDSNKIAVPFDKFLNDNFPWHVMESISDVDNENWDRYNSKIVPLSELEKTLI

SEQ ID NO: 22 - DNA, 11656 bp, Artificial
  yeast expression vector with ara A, B and D genes
gacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaaggacccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaattaaccctcactaaagggaacaaaagctgggtaccgggccccccctcgagcctaggaagccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatgtgggaggagggcgtgaatgtaagcgtgacataactaattacatgatatcgacaaaggaaaaggggatccgacgtcacctaccgtaaacgttttggtaccttttgtacagggattcaatcttagcctgatcgatgggcaatggttctcctaattgcctgctgatgtgaacagtccttgccacttcttcacacataacggcagccttcacggcctctcttgcgcttttccctatagtgaagggcccatggttctgcattagcacagccggagagtttgaattctttagtgtctcgacgattccctggcctattgaatcatctccgattaacgcaaaaggaccgacaggaatcggacccccaaactcatctcccatcatagttagaacgcagggaatttcttctcctcttgcagcccatgctgtggcataggtagagtgtgtatggacgacaccacccacttcgggcatatgtctatatacataggcatgagcagctgtgtcagaactgggacttagttcgggattgccccagtcaaccgtacctgctgatcccgtattcatacctctgacgggcgtcccatatagatcggtaacaaccattagttccggggtcaactgatcgtagctaacgccactgggtttgatcaccattaagtcatggcccggaatcctaccggatacattaccagcagtccaaacaaccagctcgtatctggtcagttctgcgtgtaagtcgcagacatctctcctgaccttggcgatagactccagaagtgaactcattttcttaagctttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaaccggtttcttcttcagattccctcatggagaaagtgcggcagatgtatatgacagagtcgccagtttccaagagactttattcaggcacttccatgataggcaagagagaagacccagagatgttgttgtcctagttacacatggtatttattccagagtattcctgatgaaatggtttagatggacatacgaagagtttgaatcgtttaccaatgttcctaacgggagcgtaatggtgatggaactggacgaatccatcaatagatacgtcctgaggaccgtgctacccaaatggactgattgtgagggagacctaactacatagtgtttaaagattacggatatttaacttacttagaataatgccatttttttgagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacgcatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaatctagattaataaaatgtcgaattatgtcatcgggcttgattacggaagtgactctgttagagcagtgctagttaacattgattccggtaaagaggaagctagttccacccatctatacaagagatggaaggaagacaaatactgtgaaccaagcataaaccagttcagacaacatccgttggatcacatagaagggcttgagaaaactataaaaagtgtgttgcaaaagaccggagttgaaggtaacagtgtgaaagccatatgcatagatactacgggatctagtccagtccctgtcaataaagacggtaaggccctagcactaacagaaggatttgaagaaaatcctaacgcaatgatggtgctgtggaaggatcacacatctatcaacgaggccaatgaaatcaatcaccttgcccgtagttgggaaggtgaagattataccaaatacgaaggaggcatctactcgtcagaatggttttgggccaagattttgcacatcgctcgtgaagatgagaaggtcaagaatgctgcatggtcatggatggaacattgtgacctgatgacatacattttgatcgggggttccgatttagagtcctttaaaaggtccaggtgtgccgcgggacataaggctatgtggcatgagtcttggggaggattacctagcaaagatttcttaagtcaactggatccttacttggccgaattaaaggatagactttatgagaagacatacacgtcagatgaagtagcaggtaatttgagcaaagaatgggctgggaaattagggctttcaactgagtgcatcatctcagttggcacctttgacgcccatgcaggtgcagtaggtgccaaaattgatgaacatagcttagtgcgtgttatgggaacatccacgtgtgacattatggtggcaagaaatgaggagataggtaaaaacacagtcaagggtatctgcggtcaagttgatggttcagtgattcctggtatgatcggactagaagcaggtcaatcagcttttggagacgtgctagcctggttcaaggacgttttgtcctggcctttagagaatctagtttacgattcagaaatactagccgaagagcaaaagaaaaagcttagagaagaagttgaagataatttcattcccaagttaacagcacaagctgagaaattagacttgagtgagtctatgcctattgctcttgattgggtaaatggtcgtcgtacccctgatgccaaccaagaattaaagtctgctattacgaatctatcgttaggtactaaagcaccccatattttcaatgctctagtaaactctatctgtttcggcagtaagatgatagttgataggtttgagtcggaaggcgtcaaaattaacaatgtaataggcataggcggcgtagctaggaagtctgcgtttattatgcagacactagccaacacattagacatgccaatcaaggtcgcaagttccgacgaagcgccagcattgggtgctgctatctacgcagcagtggctgcaggtttgtaccccaatacaatagaagccagtaaaaagttagggtcacctttcgaagctgaataccatccacaacctgagaaagttaaagaacttaagaaatatatggctgaatatagagagttggctgatttcgtggagaacaagataactcagaagaacaagcagaacgaattcgctgtttgacgtcgcgcgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacctgttgtaatcgttcttccacacgtacgttttaaacagttgatgagaacctttttcgcaagttcaaggtgctctaatttttaaaatttttacttttcgcgacacaataaagtcttcacgacgctaaactattagtgcacataatgtagttacttggacgctgttcaataatgtataaaatttatttcctttgcattacgtacattatataaccaaatcttaaaaatatagaaatatgatatgtgtataataatataagcaaaatttacgtatctttgcttataatatagctttaatgttctttaggtatatatttaagagcgatttgtctcgagagcgctacattccgtgctgaaacaagtggtagtatgcttcgttagcattcaaggtatccttaaactgccttactgacgtattgtcgtcaatcactagaagttctataccggctatgtcggcaaaatcttccaaaaattcagtcgataaggcttgagtatatactgtatgatgagctccccctgccaatatccaagcggtaacagcagtatccatgtctggttttggatcccataagacccttgccacaggtaagttaggtaaatcagcttcaggttccacggcttcgacttcgttaacgattagtctgaaacgtgttcccatatcaacaagcgatgcattcagtgctttacccttcggtgagttgaacaccaaccttactggatcttctttgcctccaatacctagcggatggacttcgcaagtaggcttactgtcagcgatactaggacatatttctaacatatgtgaacccaacacatagtccttaccttccgtaaaatgattggtgtaatcttccataaaggatgtcccaccttccatgccttgggccatgaccttcatggctcttagtagagctgctgttttccaatcaccttcagctccgaaaccataaccatcagccattaaacgttggacagcaagacccggtaattgtttcagtgcgcccagattttcgaaggtatctgtgaatgccatgaaaccaccttcttccaagaacgcacgtagtcctaattcaatcttcgcagcatcaactaagctttgtctttgatcgccaccatcctttagtgcgtcagtcaggtcgtaatctttttcatactttttcagtagtgagttaacttcatcatcgctcactttgtcgatatgttgtgtgacgtcagaggagtcgtaagcattaacttccaccccaaatttgatttgggctgcgactttatctccttcggtgacggccacttgtctcatattatccccaattctagcgaccttgatgtgctgcaattcatcccagcccaaggcaacacgttgccaatttccgactttcttttgtgtaacctctgtcttccagtggcctacaattactttcctcttcttcctcatacgggacataatgaatccaaattccctatcgccatgagccgattggttcagattcataaaatccatatcaattttggaccaggggatctcagcattaaattgggtgtgaaagtggcatataggtttcttgattatagacaacccttttatccacatctttgctggagagaaagtatgcatccataaaataaccccaatgcatgaacttgagttgttcgcatctaacatgacttttgttatctcatccgatgatttgaccgtatcttggtgaattaactttacaggtacgttatcggagccatttaaaccttcaactatttctttggaattgttagcaacttgccttaacgtttcttcgccatatagatgctgggatccggtgataaaccagacttctttattctcaaaatttgtcattttgatatctgcagaattaaaaaaactttttgtttttgtgtttattctttgttcttagaaaagacaagttgagcttgtttgttcttgatgttttattattttacaatagctgcaaatgaagaatagattcgaacattgtgaagtattggcatatatcgtctctatttatactttttttttttcagttctagtatattttgtattttcctccttttcattctttcagttgccaataagttacaggggatctcgaaagatggtggggatttttccttgaaagacgactttttgccatctaatttttccttgttgcctctgaaaattatccagcagaagcaaatgtaaaagatgaacctcagaagaacacgcaggggcccgaaattgttcctacgagaagtagccgcggccgccaccgcggtggagctccaattcgccctatagtgagtcgtattacaattcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 23 - DNA, 34 bp, Artificial
  primer DPF
aagagctcaccggtttatcattatcaatactgcc

SEQ ID NO: 24 - DNA, 44 bp, Artificial
  primer DPR
aagaattcaagctttatgtgtgtttattcgaaactaagttcttg

SEQ ID NO: 25 - DNA, 30 bp, Artificial
  primer DTF
aagaattcggatccccttttcctttgtcga

SEQ ID NO: 26 - DNA, 29 bp, Artificial
  primer DTR
aactcgagcctaggaagccttcgagcgtc

SEQ ID NO: 27 - DNA, 34 bp, Artificial
  primer AADF
aaaagcttaagaaaatgagttcacttctggagtc

SEQ ID NO: 28 - DNA, 34 bp, Artificial
  primer AADR
ttggatccgacgtcacctaccgtaaacgttttgg

SEQ ID NO: 29 - DNA, 31 bp, Artificial
  primer CMDF
aaaagcttaagaaaatgtccacgtatgcccc

SEQ ID NO: 30 - DNA, 32 bp, Artificial
  primer CMDR
ttggatccgacgtcattttaacgcaccttgcg

SEQ ID NO: 31 - DNA, 34 bp, Artificial
  primer GFDF
aaaagcttaagaaaatgtcgagccaatacaaaga

SEQ ID NO: 32 - DNA, 34 bp, Artificial
  primer GFDF
ttggatccgacgtcagttctgtccataatatgcg

SEQ ID NO: 33 - DNA, 26 bp, Artificial
  primer BPF
aaccggtttcttcttcagattccctc

SEQ ID NO: 34 - DNA, 36 bp, Artificial
  primer BPR
ttagatctctagatttatgtatgtgttttttgtagt

SEQ ID NO: 35 - DNA, 33 bp, Artificial
  primer BTF
aaagatctgcgcgcgaatttcttatgatttatg

SEQ ID NO: 36 - DNA, 28 bp, Artificial
  primer DTR
ttaagcttcgtacgtgtggaagaacgat

SEQ ID NO: 37 - DNA, 40 bp, Artificial
  primer AABF
aatctagattaataaaatgaatacgtccgaaaacataccc

SEQ ID NO: 38 - DNA, 27 bp, Artificial
  primer AABR
ttgcgcgcgacgtcacgcggacgcccc

SEQ ID NO: 39 - DNA, 32 bp, Artificial
  primer CMBF
aatctagattaataaaatgccttcggctcccg

SEQ ID NO: 40 - DNA, 34 bp, Artificial
  primer CMBR
ttgcgcgcgacgtcaggccctggcttcccttttc

SEQ ID NO: 41 - DNA, 37 bp, Artificial
  primer GFBF
aatctagattaataaaatgtcgaattatgtcatcggg

SEQ ID NO: 42 - DNA, 31 bp, Artificial
  primer GFBR
ttgcgcgcgacgtcaaacagcgaattcgttc

SEQ ID NO: 43 - DNA, 29 bp, Artificial
  primer APF
aagcggccgcggctacttctcgtaggaac

SEQ ID NO: 44 - DNA, 38 bp, Artificial
  primer APR
ttagatctgcagaattaaaaaaactttttgtttttgtg

SEQ ID NO: 45 - DNA, 36 bp, Artificial
  primer ATF
aaagatctcgagacaaatcgctcttaaatatatacc

SEQ ID NO: 46 - DNA, 36 bp, Artificial
  primer ATR
ttaagcttcgtacgttttaaacagttgatgagaacc

SEQ ID NO: 47 - DNA, 34 bp, Artificial
  primer AAAF
aactgcagatatcaaaatgccatcagctaccagc

SEQ ID NO: 48 - DNA, 34 bp, Artificial
  primer AAAR
ttctcgagagcgctaaagaccaccagctagtttg

SEQ ID NO: 49 - DNA, 33 bp, Artificial
  primer CMAF
aactgcagatatcaaaatgagcagaatcaccac

SEQ ID NO: 50 - DNA, 37 bp, Artificial
  primer CMAR
ttctcgagagcgtcataaaccttgagctaacctatgg

SEQ ID NO: 51 - DNA, 43 bp, Artificial
  primer GFAF
aactgcagatatcaaaatgacaaattttgagaataaagaagtc

SEQ ID NO: 52 - DNA, 34 bp, Artificial
  primer GFAR
ttctcgagagcgctacattccgtgctgaaacaag